(19) United States
(12) Patent Application Publication
(10) Pub. No.: US 2010/0034821 A1
(43) Pub. Date: Feb. 11, 2010

(54) GENETIC VARIATIONS ASSOCIATED WITH TUMORS

(76) Inventors: Jose F. Bazan, Menlo Park, CA (US); Joshua Kaminker, San Francisco, CA (US); Brock Peters, San Francisco, CA (US); Somasekar Seshagiri, San Carlos, CA (US); Zemin Zhang, Foster City, CA (US)

Correspondence Address: GENENTECH, INC., 1 DNA WAY, SOUTH SAN FRANCISCO, CA, 94080, US

(21) Appl. No.: 12/522,519

(22) PCT Filed: Jan. 10, 2008
(86) PCT No.: PCT/US2008/050725

§ 371 Date: Oct. 1, 2009

Related U.S. Application Data
(60) Provisional application No. 60/884,479, filed on Jan. 11, 2007.

(51) Int. Cl. A61K 39/395; C12Q 1/68; A61P 35/00

(52) U.S. Cl. 424/138.1; 435/6

(57) ABSTRACT

Tumor-associated variations in protein kinases are provided. Compositions and methods for detecting such variations and for diagnosing and treating tumors are provided.

### CROSS REFERENCE TO RELATED APPLICATION

This application claims the benefit of U.S. Provisional Application No. 60/884,479, filed Jan. 11, 2007, the disclosure of which is incorporated herein by reference in its entirety.

### FIELD OF THE INVENTION

The present invention generally relates to tumor-associated variations in protein kinases.

### BACKGROUND

Polymorphisms are heritable variations that are present in an organism's genome. Polymorphisms include restriction fragment length polymorphisms (RFLPs), short tandem repeats (STRs), and single nucleotide polymorphisms (SNPs). SNPs in particular may be associated with susceptibility to certain diseases, including cancer. (See, e.g., Zhu et al. (2004) Cancer Research 64:2251-2257.) Thus, a continuing need exists to identify polymorphisms associated with cancer.

Tyrosine kinase 2, or “TYK2,” is a member of the Janus, or “Jak,” family of non-receptor tyrosine kinases, which also includes JAK1, JAK2, and JAK3. Jak kinases are generally large proteins (about 1100 amino acids) that share a common structure comprising several domains. Those domains include a C-terminal kinase domain, a “pseudokinase” domain and a “FERM” domain, with the latter two domains apparently interacting with and regulating the catalytic activity of the kinase domain. For review, see, e.g., Yamaoka et al. (2004) Genome Biol. 5:253.

Jak kinases associate with cytokine and hormone receptors and become activated upon binding of ligand to those receptors. Activated Jaks phosphorylate their associated receptors and other substrates. The phosphorylated receptors bind to signal transducers and activators of transcription (STATs). Constitutive activation of JAK1, JAK2, and certain STATs has been correlated with cancer. See, e.g., supra; Verma et al. (2003) Cancer and Metastasis Rev. 22:423-434. Thus, a continuing need exists to identify TYK2 variants (e.g., polymorphic variants) associated with cancer.

The invention described herein meets the above-described needs and provides other benefits.

### SUMMARY

The compositions and methods of the invention are based, at least in part, on the discovery of tumor-associated variations in protein kinases. In one example, a novel germline mutation in the TYK2 gene has been discovered in four independent human tumor tissue samples. That mutation results in an amino acid substitution in the catalytic kinase domain. In further examples, tumor-associated variations have been discovered in two other human kinases, MAST2 and RIOK2.

In one aspect of the invention, a method of diagnosing a tumor in a patient is provided, the method comprising detecting the presence of an amino acid variation in the catalytic kinase domain of TYK2 in a biological sample derived from the patient. In one embodiment, the method comprises detecting the presence of an amino acid variation at P1104, P1105, or W1067 of TYK2 in the biological sample. In one such embodiment, the amino acid variation is an amino acid substitution at P1104. In one such embodiment, the amino acid substitution is P1104A. In another embodiment, the detecting comprises detecting the presence of a nucleotide variation in a TYK2 polynucleotide derived from the biological sample, wherein the nucleotide variation results in the amino acid variation. In one such embodiment, the nucleotide variation is a C3651G substitution. In another such embodiment, the nucleotide variation in the TYK2 polynucleotide comprises a nucleotide variation in SEQ ID NO:1 or a coding region thereof. In another such embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay. In another embodiment, the biological sample comprises or is suspected of comprising tumor cells. In another embodiment, the tumor is a breast, colon, or stomach tumor.

In another aspect, a method is provided for determining whether a tumor in a patient will respond to a therapeutic agent that targets a TYK2 or TYK2 polynucleotide, the method comprising detecting the absence or presence of an amino acid variation in the catalytic kinase domain of TYK2 in a biological sample derived from the patient, wherein the presence of the amino acid variation indicates that the tumor will respond to the therapeutic agent. In one embodiment, the method comprises detecting the absence or presence of an amino acid variation at P1104, P1105, or W1067 of TYK2 in the biological sample. In one such embodiment, the amino acid variation is an amino acid substitution at P1104. In one such embodiment, the amino acid substitution is P1104A. In another embodiment, the tumor is selected from a breast, colon, or stomach tumor. In another embodiment, the amino acid variation is detected by detecting a nucleotide variation in a TYK2 polynucleotide derived from the biological sample, wherein the nucleotide variation results in the amino acid variation. In one such embodiment, the nucleotide variation is a C3651G substitution. In another such embodiment, the nucleotide variation in the TYK2 polynucleotide comprises a nucleotide variation in SEQ ID NO:1 or a coding region thereof. In another such embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In another aspect, a method of inhibiting the proliferation of a tumor cell is provided, wherein the tumor cell comprises a TYK2 having an amino acid variation in the catalytic kinase domain, the method comprising exposing the tumor cell to an antagonist of TYK2. In one embodiment, the amino acid variation occurs at P1104, P1105, or W1067 of TYK2. In another embodiment, the amino acid variation is an amino acid substitution at P1104. In one such embodiment, the amino acid substitution is P1104A. In another embodiment, the tumor cell is a breast, colon, or stomach tumor cell. In another embodiment, the antagonist of TYK2 is a small molecule antagonist. In another embodiment, the antagonist of TYK2 is an antagonist antibody. In another embodiment, the method further comprises administering a cytotoxic agent, chemotherapeutic agent, or growth inhibitory agent to the patient.

In another aspect, a method of treating a tumor comprising a TYK2 having an amino acid variation in the catalytic kinase domain is provided, the method comprising administering to a patient having the tumor an effective amount of a pharmaceutical formulation comprising an antagonist of TYK2. In one embodiment, the amino acid variation occurs at P1104, P1105, and W1067. In one such embodiment, the amino acid variation is an amino acid substitution at P1104. In one such embodiment, the amino acid substitution is P1104A. In another embodiment, the tumor is a breast, colon, or stomach tumor. In another embodiment, the antagonist of TYK2 is a small molecule antagonist. In another embodiment, the antagonist of TYK2 is an antagonist antibody. In another embodiment, the method further comprises administering a cytotoxic agent, chemotherapeutic agent, or growth inhibitory agent to the patient.

In another aspect, an isolated polynucleotide is provided, wherein the isolated polynucleotide comprises (a) a TYK2 polynucleotide or fragment thereof that is at least about 10 nucleotides in length, wherein the TYK2 polynucleotide or fragment thereof comprises a nucleotide variation that results in an amino acid variation at P1104, P1105, or W1067 of TYK2, or (b) the complement of (a). In one embodiment, the amino acid variation is an amino acid substitution at P1104. In one such embodiment, the amino acid substitution is P1104A. In another embodiment, the nucleotide variation is a C3651G substitution. In another embodiment, the TYK2 polynucleotide or fragment thereof comprises a nucleotide variation in the sequence of SEQ ID NO:1 or a fragment thereof. In another embodiment, the isolated polynucleotide is a primer. In another embodiment, the isolated polynucleotide is an oligonucleotide.

In another aspect, an oligonucleotide is provided that is (a) an allele-specific oligonucleotide that hybridizes to a region of a TYK2 polynucleotide comprising a nucleotide variation that results in an amino acid variation at P1104, P1105, or W1067 of TYK2, or (b) the complement of (a). In one embodiment, the amino acid variation is an amino acid substitution at P1104. In one such embodiment, the amino acid substitution is P1104A. In another embodiment, the nucleotide variation is a C3651G substitution. In another embodiment, the TYK2 polynucleotide comprises a nucleotide variation in the sequence of SEQ ID NO:1 or a coding region thereof. In another embodiment, the allele-specific oligonucleotide is an allele-specific primer. In another embodiment, a diagnostic kit comprising the oligonucleotide and at least one enzyme is provided. In one such embodiment, the at least one enzyme is a polymerase. In another such embodiment, the at least one enzyme is a ligase. In another embodiment, a microarray comprising the oligonucleotide is provided.

In another aspect, a method of detecting a tumor in a biological sample is provided, the method comprising detecting the presence of a nucleotide variation in a TYK2 polynucleotide derived from the biological sample, wherein the nucleotide variation results in an amino acid variation at P1104, P1105, or W1067 of TYK2. In one embodiment, the amino acid variation is an amino acid substitution at P1104. In one such embodiment, the amino acid substitution is P1104A. In another embodiment, the nucleotide variation is a C3651G substitution. In another embodiment, the nucleotide variation in the TYK2 polynucleotide comprises a nucleotide variation in SEQ ID NO:1 or a coding region thereof. In another embodiment, the tumor is selected from a breast, colon, or stomach tumor. In another embodiment, the detecting comprises carrying out a process selected from a primer extension assay; an allele-specific primer extension assay; an allele-specific nucleotide incorporation assay; an allele-specific oligonucleotide hybridization assay; a 5′ nuclease assay; an assay employing molecular beacons; and an oligonucleotide ligation assay.

In another aspect, a method of detecting the presence of a nucleotide variation in a TYK2 polynucleotide is provided, wherein the nucleotide variation results in an amino acid variation at P1104, P1105, or W1067 of TYK2, the method comprising (a) contacting nucleic acid suspected of comprising the nucleotide variation with an allele-specific oligonucleotide that is specific for the nucleotide variation under conditions suitable for hybridization of the allele-specific oligonucleotide to the nucleic acid; and (b) detecting the presence of allele-specific hybridization, wherein the presence of allele-specific hybridization indicates the presence of a nucleotide variation in a TYK2 polynucleotide. In one embodiment, the amino acid variation is an amino acid substitution at P1104. In one such embodiment, the amino acid substitution is P1104A. In another embodiment, the nucleotide variation is a C3651G substitution. In another embodiment, the nucleotide variation in the TYK2 polynucleotide comprises a nucleotide variation in SEQ ID NO:1 or a coding region thereof.

In another aspect, a method of amplifying a nucleic acid comprising a nucleotide variation is provided, wherein the nucleotide variation results in an amino acid variation at P1104, P1105, or W1067 of TYK2, the method comprising (a) contacting the nucleic acid with a primer that hybridizes to a sequence 3′ of the nucleotide variation, and (b) extending the primer to generate an amplification product comprising the nucleotide variation. In one embodiment, the amino acid variation is an amino acid substitution at P1104. In one such embodiment, the amino acid substitution is P1104A. In another embodiment, the nucleotide variation is a C3651G substitution. In another embodiment, the nucleic acid comprises a nucleotide variation in SEQ ID NO:1 or a fragment thereof.

In further aspects, compositions and methods related to the discovery of tumor-associated variations in TYK2 and the serine-threonine kinases MAST2 and RIOK2 are provided as described below.

### BRIEF DESCRIPTION OF THE FIGURES

FIG. 1 shows sequence trace files for the MAST2 (A) and RIOK2 (B) genes. Both forward and reverse traces are shown as labeled. The arrow in each panel indicates the position of the variation. Nucleotides are indicated at the top of each histogram with the N representing the wild-type/mutant nucleotides of A/G (panel A) or T/C (panel B). Trace data for 10 base pairs flanking either side of the variant are shown and indicate the high quality of reliable sequence for these regions.

FIG. 2 shows the identification and characterization of a cancer-associated germline mutation in human TYK2 (NM_{—}003331.3; SEQ ID NO:1 (nucleotide sequence) and SEQ ID NO:2 (amino acid sequence)). (A) A mass spectrometry method based on the Sequenom platform was used to screen for the C3651G (P1104A) mutation in genomic DNA from unrelated tumor samples. A representative positive mass spectrum is shown to indicate the presence of the heterozygous C/G in a tumor tissue sample. This variant was originally observed in 4 EST clones from 3 cancer libraries. None of the 29 EST sequences from normal libraries covering this position contain this mutation. The table in panel (B) shows the occurrence and frequency of the TYK2 mutation observed in screened tumor samples. In all 4 cases where the C3651G mutation was found, the same mutation was also found in the matched normal tissues, indicating that the mutation is a germline mutation. The predicted structure of the TYK2 kinase domain is shown in panel (C). The TYK2 model was generated using the program Modeller using the X-ray structures of the Jak2 and Jak3 kinase domains (PDB files 2B7A and 1YVJ, respectively) as templates. Proline 1104 is indicated in black in the boxed regions, while the interacting proline 1105 and tryptophan 1067 residues are in light grey in the boxed regions. The mutated P1104 residue lies in the substrate-binding groove of TYK2, and it closely packs in a ring-stacking interaction with a conserved W1067 residue tied to the activation loop. P1104 also contacts the ring of the neighboring P1105 residue which helps position the adjacent α-helix.

### DETAILED DESCRIPTION OF EMBODIMENTS

### I. Definitions

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O— allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and allowing the polymerization of a complementary nucleic acid, generally by providing a free 3′-OH group.

The term “TYK2,” as used herein, refers to any native tyrosine kinase 2 from any vertebrate source, including mammals such as primates (e.g. humans and monkeys) and rodents (e.g., mice and rats), unless otherwise indicated. The term encompasses “full-length,” unprocessed TYK2 as well as any form of TYK2 that results from processing in the cell. The term also encompasses naturally occurring variants of TYK2, e.g., splice variants, allelic variants, and other isoforms. The term also encompasses fragments or variants of a native TYK2 that maintain at least one biological activity of TYK2, e.g., kinase activity.

The term “TYK2 polynucleotide” refers to a gene or coding sequence (e.g., an RNA or cDNA coding sequence) that encodes a TYK2, unless otherwise indicated.

The term “MAST2,” as used herein, refers to any microtubule-associated serine/threonine kinase 2 from any vertebrate source, including mammals such as primates (e.g. humans and monkeys) and rodents (e.g., mice and rats), unless otherwise indicated. The term encompasses “full-length,” unprocessed MAST2 as well as any form of MAST2 that results from processing in the cell. The term also encompasses naturally occurring variants of MAST2, e.g., splice variants, allelic variants, and other isoforms. The term also encompasses fragments or variants of a native MAST2 that maintain at least one biological activity of MAST2, e.g., kinase activity.

The term “MAST2 polynucleotide” refers to a gene or coding sequence (e.g., an RNA or cDNA coding sequence) that encodes a MAST2, unless otherwise indicated.

The term “RIOK2,” as used herein, refers to any native serine-threonine protein kinase RIO2 from any vertebrate source, including mammals such as primates (e.g. humans and monkeys) and rodents (e.g., mice and rats), unless otherwise indicated. The term encompasses “full-length,” unprocessed RIOK2 as well as any form of RIOK2 that results from processing in the cell. The term also encompasses naturally occurring variants of RIOK2, e.g., splice variants, allelic variants, and other isoforms. The term also encompasses fragments or variants of a native RIOK2 that maintain at least one biological activity of RIOK2 e.g., kinase activity.

The term “RIOK2 polynucleotide” refers to a gene or coding sequence (e.g., an RNA or cDNA coding sequence) that encodes a RIOK2, unless otherwise indicated.

The term “PRO” refers to any of TYK2, MAST2, or RIOK2, unless otherwise indicated.

The term “PRO polynucleotide” refers to a gene or coding sequence (e.g., an RNA or cDNA coding sequence) that encodes a PRO, unless otherwise indicated.

The term “variation” refers to either a nucleotide variation or an amino acid variation.

The term “nucleotide variation” refers to a change in a polynucleotide sequence (e.g., a nucleotide insertion, deletion, inversion, or substitution, such as a single nucleotide polymorphism (SNP)) relative to a reference polynucleotide sequence (e.g., a wild type sequence). The term also encompasses the corresponding change in the complement of the polynucleotide sequence, unless otherwise indicated. A nucleotide variation may be a germline or somatic variation.

The term “amino acid variation” refers to a change at a specified amino acid position(s) in a polypeptide sequence (e.g., a change caused by a deletion, insertion, or substitution of one or more amino acids) relative to a reference polypeptide sequence (e.g., a wild type sequence).

The term “activating variation” refers to a variation in a gene or gene product that results in a more active form of the gene product, relative to the wild type gene product.

The term “array” or “microarray” refers to an ordered arrangement of hybridizable array elements, preferably polynucleotide probes (e.g., oligonucleotides), on a substrate. The substrate can be a solid substrate, such as a glass slide, or a semi-solid substrate, such as nitrocellulose membrane.

The term “amplification” refers to the process of producing one or more copies of a reference nucleic acid sequence or its complement. Amplification may be linear or exponential (e.g., PCR). A “copy” does not necessarily mean perfect sequence complementarity or identity relative to the template sequence. For example, copies can include nucleotide analogs such as deoxyinosine, intentional sequence alterations (such as sequence alterations introduced through a primer comprising a sequence that is hybridizable, but not fully complementary, to the template), and/or sequence errors that occur during amplification.

The term “allele-specific oligonucleotide” refers to an oligonucleotide that hybridizes to a region of a target nucleic acid that comprises a nucleotide variation (generally a nucleotide substitution). “Allele-specific hybridization” means that, when an allele-specific oligonucleotide is hybridized to its target nucleic acid, a nucleotide in the allele-specific oligonucleotide specifically base pairs with the variation. An allele-specific oligonucleotide capable of allele-specific hybridization with respect to a particular variation is said to be “specific for” that variation.

The term “allele-specific primer” refers to an allele specific oligonucleotide that is a primer.

The term “primer extension assay” refers to an assay in which nucleotides are added to a nucleic acid, resulting in a longer nucleic acid, or “extension product,” that is detected directly or indirectly.

The term “allele-specific nucleotide incorporation assay” refers to a primer extension assay in which a primer is (a) hybridized to target nucleic acid at a region that is 3′ of a nucleotide variation and (b) extended by a polymerase, thereby incorporating into the extension product a nucleotide that is complementary to a variation.

The term “allele-specific primer extension assay” refers to a primer extension assay in which an allele-specific primer is hybridized to a target nucleic acid and extended.

The term “allele-specific oligonucleotide hybridization assay” refers to an assay in which (a) an allele-specific oligonucleotide is hybridized to a target nucleic acid and (b) hybridization is detected directly or indirectly.

The term “5′nuclease assay” refers to an assay in which hybridization of an allele-specific oligonucleotide to a target nucleic acid is followed by nucleolytic cleavage of the hybridized probe, resulting in a detectable signal.

The term “assay employing molecular beacons” refers to an assay in which hybridization of an allele-specific oligonucleotide to a target nucleic acid results in a level of detectable signal that is higher than the level of detectable signal emitted by the free oligonucleotide.

The term “oligonucleotide ligation assay” refers to an assay in which an allele-specific oligonucleotide and a second oligonucleotide are hybridized adjacent to one another on a target nucleic acid and ligated together, and the ligation product is detected directly or indirectly.

The term “target sequence,” “target nucleic acid,” or “target nucleic acid sequence” refers generally to a polynucleotide sequence of interest in which a variation is suspected or known to reside, including copies of such target nucleic acid generated by amplification.

The term “detection” includes any means of detecting, including direct and indirect detection.

The term “diagnosis” is used herein to refer to the identification of a molecular or pathological state, disease or condition, such as the identification of a cancer (e.g., a colon cancer) or a particular type of cancer (e.g., a colon cancer characterized by a particular variation). The term “prognosis” is used herein to refer to the prediction of the likelihood of cancer-attributable death or progression, including, for example, recurrence, metastatic spread, and drug resistance, of a neoplastic disease, such as cancer. The term “prediction” is used herein to refer to the likelihood that a patient will respond either favorably or unfavorably to a drug or set of drugs. In one embodiment, the prediction relates to the extent of those responses. In one embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The terms “cell proliferative disorder” and “proliferative disorder” refer to disorders that are associated with a measurable degree of abnormal cell proliferation. In one embodiment, the cell proliferative disorder is cancer.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer, colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

The term “colorectal tumor” or “colorectal cancer” refers to any tumor or cancer of the large bowel, which includes the colon (the large intestine from the cecum to the rectum) and the rectum, including, e.g., adenocarcinomas and less prevalent forms, such as lymphomas and squamous cell carcinomas.

The term “colorectal tumor cell” or “colorectal cancer cell” refers to a colorectal tumor cell or colorectal cancer cell, either in vivo or in vitro, and encompasses cell lines derived from such cells.

The term “colon tumor” or “colon cancer” refers to any tumor or cancer of the colon (the large intestine from the cecum to the rectum).

The term “colon tumor cell” or “colon cancer cell” refers to a colon tumor cell or colon cancer cell, either in vivo or in vitro, and encompasses cell lines derived from such cells.

The term “stomach tumor” or “stomach cancer” refers to any tumor or cancer of the stomach, including, e.g., adenocarcinomas (such as diffuse type and intestinal type), and less prevalent forms such as lymphomas, leiomyosarcomas, and squamous cell carcinomas.

The term “stomach tumor cell” or “stomach cancer cell” refers to a stomach tumor cell or stomach cancer cell, either in vivo or in vitro, and encompasses cell lines derived from such cells.

The term “breast tumor” or “breast cancer” refers to any tumor or cancer of the breast, including, e.g., adenocarcinomas, such as invasive or in situ ductal carcinoma, invasive or insitu lobular carcinoma, medullary carcinoma, colloid carcinoma, and papillary carcinoma; and less prevalent forms, such as cytosarcoma phylloides, sarcomas, squamous cell carcinomas, and carcinosarcomas.

The term “breast tumor cell” or “breast cancer cell” refers to a breast tumor cell or breast cancer cell, either in vivo or in vitro, and encompasses cell lines derived from such cells.

The term “neoplasm” or “neoplastic cell” refers to an abnormal tissue or cell that proliferates more rapidly than corresponding normal tissues or cells and continues to grow after removal of the stimulus that initiated the growth.

A “tumor cell” or “cancer cell” refers to a tumor cell or cancer cell, either in vivo or in vitro, and encompasses cell lines derived from such cells.

As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “individual,” “subject,” or “patient” is a vertebrate. In certain embodiments, the vertebrate is a mammal. Mammals include, but are not limited to, farm animals (such as cows), sport animals, pets (such as cats, dogs, and horses), primates (including human and non-human primates), and rodents (e.g., mice and rats). In certain embodiments, a mammal is a human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The term “long-term” survival is used herein to refer to survival for at least 1 year, 5 years, 8 years, or 10 years following therapeutic treatment.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A tumor that “responds” to a therapeutic agent is one that shows any decrease in tumor progression, including but not limited to, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; and/or (5) inhibition (i.e. reduction, slowing down or complete stopping) of metastasis.

The term “antagonist” is used in the broadest sense, and includes any molecule that partially or fully inhibits or neutralizes a biological activity (e.g., kinase activity) of a polypeptide (e.g., PRO), or that partially or fully inhibits the transcription or translation of a nucleic acid encoding the polypeptide. Suitable antagonist molecules include, but are not limited to, antagonist antibodies, polypeptide fragments, oligopeptides, organic molecules (including small molecules), and anti-sense nucleic acids.

The term “agonist” is used in the broadest sense, and includes any molecule that partially or fully mimics a biological activity of a polypeptide, or that increases the transcription or translation of a nucleic acid encoding the polypeptide. Suitable agonist molecules include, but are not limited to, agonist antibodies, polypeptide fragments, oligopeptides, organic molecules (including small molecules), polynucleotides, polypeptides, and polypeptide-Fc fusions.

A “therapeutic agent that targets a PRO or a PRO polynucleotide” means any agent that affects the expression and/or activity of PRO or a PRO polynucleotide including, but not limited to, any of the PRO antagonists described herein, including such therapeutic agents that are already known in the art as well as those that are later developed.

The term “cytotoxic agent” as used herein refers to a substance that inhibits or prevents a cellular function and/or causes cell death or destruction. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu), chemotherapeutic agents (e.g., methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A “tumoricidal” agent causes destruction of tumor cells.

A “toxin” is any substance capable of having a detrimental effect on the growth or proliferation of a cell.

A “chemotherapeutic agent” is a chemical compound useful in the treatment of cancer.

Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell (such as a cell expressing TYK2) either in vitro or in vivo. Thus, the growth inhibitory agent may be one which significantly reduces the percentage of cells (such as a cell expressing PRO) in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “anti-PRO antibody” or “an antibody that binds to PRO” refers to an antibody that is capable of binding PRO with sufficient affinity such that the antibody is useful as a diagnostic and/or therapeutic agent in targeting PRO. Preferably, the extent of binding of an anti-PRO antibody to an unrelated, non-PRO protein is less than about 10% of the binding of the antibody to PRO as measured, e.g., by a radioimmunoassay (RIA). In certain embodiments, an antibody that binds to PRO has a dissociation constant (Kd) of ≦1 μM, ≦100 nM, ≦10 nM, ≦1 nM, or ≦0.1 nM. In certain embodiments, an anti-PRO antibody binds to an epitope of PRO that is conserved among PRO from different species.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region.

“Antibody fragments” comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example, one that comprises the Fc region, retains at least one of the biological functions normally associated with the Fc region when present in an intact antibody, such as FcRn binding, antibody half life modulation, ADCC function and complement binding. In one embodiment, an antibody fragment is a monovalent antibody that has an in vivo half life substantially similar to an intact antibody. For example, such an antibody fragment may comprise an antigen binding arm linked to an Fc sequence capable of conferring in vivo stability to the fragment.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is a minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment contains the heavy- and light-chain variable domains and also contains the constant domain of the light chain and the first constant domain (CHI) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO93/1161; Hudson et al. (2003) Nat. Med. 9:129-134; and Hollinger et al., Proc. Natl. Acad. Sci. USA 90: 6444-6448 (1993). Triabodies and tetrabodies are also described in Hudson et al. (2003) Nat. Med. 9:129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256: 495 (1975); Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol., 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci. USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

An “affinity matured” antibody is one with one or more alterations in one or more CDRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

A “blocking antibody” or an “antagonist antibody” is one which inhibits or reduces a biological activity of the antigen it binds. Certain blocking antibodies or antagonist antibodies partially or completely inhibit the biological activity of the antigen.

A “small molecule” or “small organic molecule” is defined herein as an organic molecule having a molecular weight below about 500 Daltons.

An “PRO-binding oligopeptide” or an “oligopeptide that binds PRO” is an oligopeptide that is capable of binding PRO with sufficient affinity such that the oligopeptide is useful as a diagnostic and/or therapeutic agent in targeting PRO. In certain embodiments, the extent of binding of a PRO-binding oligopeptide to an unrelated, non-PRO protein is less than about 10% of the binding of the PRO-binding oligopeptide to PRO as measured, e.g., by a surface plasmon resonance assay. In certain embodiments, a PRO-binding oligopeptide has a dissociation constant (Kd) of ≦1 μM, ≦100 nM, ≦10 nM, ≦1 nM, or ≦0.1 nM.

An “PRO-binding organic molecule” or “an organic molecule that binds PRO” is an organic molecule other than an oligopeptide or antibody as defined herein that is capable of binding PRO with sufficient affinity such that the organic molecule is useful as a diagnostic and/or therapeutic agent in targeting PRO. In certain embodiments, the extent of binding of a PRO-binding organic molecule to an unrelated, non-PRO protein is less than about 10% of the binding of the PRO-binding organic molecule to PRO as measured, e.g., by a surface plasmon resonance assay. In certain embodiments, a PRO-binding organic molecule has a dissociation constant (Kd) of ≦1 μM, ≦100 nM, ≦10 nM, ≦1 nM, or ≦0.1 nM.

The dissociation constant (Kd) of any molecule that binds a target polypeptide may conveniently be measured using a surface plasmon resonance assay. Such assays may employ a BIAcore™-2000 or a BIAcore™-3000 (BIAcore, Inc., Piscataway, N.J.) at 25° C. with immobilized target polypeptide CM5 chips at ˜10 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Target polypeptide is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of target polypeptide, 1 M ethanolamine is injected to block unreacted groups. For kinetics measurements, two-fold serial dilutions of the binding molecule (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneously fitting the association and dissociation sensorgrams. The equilibrium dissociation constant (Kd) is calculated as the ratio k_{off}/kₒₙ. See, e.g., Chen, Y., et al., (1999) J. Mol. Biol. 293:865-881. If the on-rate of an antibody exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

A “liposome” is a small vesicle composed of various types of lipids, phospholipids and/or surfactant which is useful for delivery of an agent, e.g., a drug, to a mammal. The components of the liposome are commonly arranged in a bilayer formation, similar to the lipid arrangement of biological membranes.

The word “label” when used herein refers to a detectable compound or composition. The label may be detectable by itself (e.g., radioisotope labels or fluorescent labels) or, in the case of an enzymatic label, may catalyze chemical alteration of a substrate compound or composition which results in a detectable product. Radionuclides that can serve as detectable labels include, for example, I-131, I-123, I-125, Y-90, Re-188, Re-186, At-211, Cu-67, Bi-212, and Pd-109.

An “isolated” biological molecule, such as a nucleic acid, polypeptide, or antibody, is one which has been identified and separated and/or recovered from at least one component of its natural environment.

### II. Description of Certain Embodiments

Methods and compositions for the diagnosis and treatment of tumors are provided. The methods and compositions of the invention are based, at least in part, on the discovery of novel tumor-associated variations in protein kinases.

The variations provided herein serve as biomarkers for cancer and/or predispose or contribute to tumorigenesis or tumor promotion. Accordingly, the variations disclosed herein are useful in a variety of settings, e.g., in methods and compositions related to cancer diagnosis and therapy.

A. TYK2 Variations

In one example, a novel germline mutation in the human TYK2 gene has been identified. That mutation results in a P104A substitution in TYK2. Proline 1104 falls within the catalytic kinase domain of TYK2, which extends from about amino acid 897 to about amino acid 1176. Specifically, P1104 lies in the substrate-binding groove of TYK2 and closely interacts with W1067 and P1105. Mutation of P1104 or the residue(s) with which it interacts may result in an activated, oncogenic form of TYK2, as described further herein.

1. Compositions

In one aspect, an isolated polynucleotide comprising at least a fragment of a TYK2 polynucleotide is provided, wherein the fragment comprises a nucleotide variation that results in an amino acid variation at P1104, P1105, or W1067 of TYK2. In one embodiment, the amino acid variation is an amino acid substitution at P104. In one such embodiment, the amino acid substitution is P1104A. Amino acid positions designated herein refer to amino acid positions in the human TYK2 amino acid sequence shown in SEQ ID NO:2. The designated amino acid positions also refer to the corresponding amino acid positions in fragments or variants of SEQ ID NO:2 (e.g., allelic variants or splice variants), which can be routinely identified using sequence alignments or similar techniques.

In another embodiment, a TYK2 polynucleotide or fragment thereof comprises a nucleotide variation in the sequence of SEQ ID NO:1 or a fragment of SEQ ID NO:1. In one embodiment, a fragment of SEQ ID NO:1 is the coding region of SEQ ID NO:1 from nucleotides 342-3905. In another embodiment, the nucleotide variation is a C3651G substitution. The nucleotide positions designated herein refer to nucleotide positions in the human TYK2 polynucleotide sequence shown in SEQ ID NO:1. The designated nucleotide positions also refer to the corresponding nucleotide positions in fragments or variants of SEQ ID NO:1 (e.g., allelic variants or splice variants), which can be routinely identified using sequence alignments or similar techniques.

In another embodiment, a fragment of a TYK2 polynucleotide is at least about 10 nucleotides in length, alternatively at least about 15 nucleotides in length, alternatively at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length, alternatively at least about 1000 nucleotides in length, and alternatively about the length of the full-length coding sequence. In this context the term “about” means the referenced nucleotide sequence length plus or minus 10% of that referenced length. In another embodiment, a TYK2 polynucleotide comprises the nucleic acid sequence of SEQ ID NO:1 or a coding region thereof. In another embodiment, the complement of any of the above polynucleotides is provided. In another embodiment, a TYK2 encoded by the any of the above polynucleotides is provided.

In one embodiment, an isolated polynucleotide provided herein is detectably labeled, e.g., with a radioisotope, a fluorescent agent, or a chromogenic agent. In another embodiment, an isolated polynucleotide is a primer. In another embodiment, an isolated polynucleotide is an oligonucleotide, e.g., an allele-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Oligonucleotides as provided herein are useful, e.g., as hybridization probes for the detection of tumor-associated variations.

In another aspect, an allele-specific oligonucleotide is provided that hybridizes to a region of a TYK2 polynucleotide comprising a nucleotide variation (e.g., a substitution) that results in an amino acid variation at P1104, P1105, or W1067 of TYK2. The allele-specific oligonucleotide, when hybridized to the region of the TYK2 polynucleotide, comprises a nucleotide that base pairs with the nucleotide variation. In one embodiment, the amino acid variation is an amino acid substitution at P1104. In one such embodiment, the amino acid substitution is P1104A. In another embodiment, the nucleotide variation is C3651G. In another embodiment, the complement of an allele-specific oligonucleotide is provided. In another embodiment, a microarray comprises an allele-specific oligonucleotide or its complement. In another embodiment, an allele-specific oligonucleotide or its complement is an allele-specific primer.

An allele-specific oligonucleotide can be used in conjunction with a control oligonucleotide that is identical to the allele-specific oligonucleotide, except that the nucleotide that specifically base pairs with the nucleotide variation is replaced with a nucleotide that specifically base pairs with the corresponding nucleotide present in the wild type TYK2 polynucleotide. Such oligonucleotides may be used in competitive binding assays under hybridization conditions that allow the oligonucleotides to distinguish between a TYK2 polynucleotide comprising a nucleotide variation and a TYK2 polynucleotide comprising the corresponding wild type nucleotide. Using routine methods based on, e.g., the length and base composition of the oligonucleotides, one skilled in the art can arrive at suitable hybridization conditions under which (a) an allele-specific oligonucleotide will preferentially bind to a TYK2 polynucleotide comprising a nucleotide variation relative to a wild type TYK2 polynucleotide, and (b) the control oligonucleotide will preferentially bind to a wild type TYK2 polynucleotide relative to a TYK2 polynucleotide comprising a nucleotide variation. Exemplary conditions include conditions of high stringency, e.g., hybridization conditions of 5× standard saline phosphate EDTA (SSPE) and 0.5% NaDodSO₄ (SDS) at 55° C., followed by washing with 2×SSPE and 0.1% SDS at 55° C. or room temperature.

In another aspect, a binding agent is provided that preferentially binds to a TYK2 comprising an amino acid variation at P1104, P1105, or W1067, relative to a TYK2 encoded by a wild-type TYK2 polynucleotide. In one embodiment, the amino acid variation is an amino acid substitution at P1104. In one such embodiment, the amino acid substitution is P1104A. In another embodiment, the binding agent is an antibody. In another embodiment, the binding agent is an oligopeptide that binds TYK2 comprising an amino acid variation at P1104, P1105, or W1067.

In another aspect, diagnostic kits are provided. In one embodiment, a kit comprises any of the foregoing polynucleotides and an enzyme. In one embodiment, the enzyme is at least one enzyme selected from a nuclease, a ligase, and a polymerase.

2. Methods

In one aspect, a method of detecting the presence of a tumor in a biological sample derived from a patient is provided, the method comprising detecting an amino acid variation in the catalytic kinase domain of TYK2 in the biological sample. In one embodiment, the amino acid variation occurs at P1104, P1105, or W1067. Detecting an amino acid variation encompasses detecting the amino acid variation directly (e.g., with an antibody that preferentially recognizes a polypeptide comprising the amino acid variation relative to the corresponding wild-type polypeptide, or vice versa) or indirectly (e.g., by detecting a nucleotide variation in a polynucleotide that encodes a polypeptide comprising the amino acid variation). In one embodiment, the amino acid variation is an amino acid substitution at P1104. In one such embodiment, the amino acid substitution is P1104A. In another embodiment, the amino acid variation is detected by detecting a nucleotide variation in the biological sample that results in the amino acid variation. In one such embodiment, the nucleotide variation is a C3651G substitution. In another such embodiment, the nucleotide variation comprises a nucleotide variation in SEQ ID NO:1 or a coding region thereof. In another embodiment, the biological sample is a biopsy (e.g., a tissue sample containing cells suspected of being cancerous). In another embodiment, the tumor is a breast, colon, or stomach tumor.

In another aspect, a method of diagnosing a tumor in a patient is provided, the method comprising detecting the presence of an amino acid variation in the catalytic kinase domain of TYK2 in a biological sample derived from the patient. In one embodiment, the amino acid variation occurs at P1104, P1105, or W1067. In another embodiment, the amino acid variation is an amino acid substitution at P1104. In one such embodiment, the amino acid substitution is P1104A. In another embodiment, the biological sample comprises or is suspected of comprising tumor cells. In one such embodiment, the tumor cells are selected from breast, colon, or stomach tumor cells. In another embodiment, the amino acid variation is detected by detecting a nucleotide variation in the biological sample that results in the amino acid variation. In one such embodiment, the nucleotide variation is a C3651G substitution. In another such embodiment, the nucleotide variation comprises a nucleotide variation in SEQ ID NO:1 or a coding region thereof.

In another aspect, a method is provided for determining whether a tumor in a patient will respond to a therapeutic agent that targets a TYK2 or TYK2 polynucleotide, the method comprising detecting the absence or presence of an amino acid variation in the catalytic kinase domain of TYK2 in a biological sample derived from the patient, wherein the presence of the amino acid variation indicates that the tumor will respond to the therapeutic agent. In one embodiment, the amino acid variation occurs at P1104, P1105, or W1067. In one such embodiment, the amino acid variation is an amino acid substitution at P1104. In one such embodiment, the amino acid substitution is P1104A. In another embodiment, the amino acid variation is detected by detecting a nucleotide variation in a TYK2 polynucleotide derived from the biological sample, wherein the nucleotide variation results in the amino acid variation. In one such embodiment, the nucleotide variation is a C3651G substitution. In another such embodiment, the nucleotide variation in the TYK2 polynucleotide comprises a nucleotide variation in SEQ ID NO:1 or a coding region thereof. In another embodiment, the tumor is a breast, colon, or stomach tumor.

In another aspect, a method of detecting the absence or presence of an amino acid variation at P1104, P1105, or W1067 of TYK2 in a biological sample is provided, the method comprising (a) contacting nucleic acid derived from the biological sample with any of the above-described polynucleotides under conditions suitable for formation of a hybridization complex between the polynucleotide and the nucleic acid; and (b) detecting whether at least one nucleotide in the nucleic acid specifically base pairs with the polynucleotide in the hybridization complex, wherein the at least one nucleotide is suspected of comprising a nucleotide variation that results in the amino acid variation, and wherein the presence of specific base pairing indicates the presence of the amino acid variation. In one embodiment, the amino acid variation is an amino acid substitution at P104. In one such embodiment, the amino acid substitution is P1104A. In another embodiment, the nucleotide variation is a C3651G substitution. In another embodiment, the nucleotide variation comprises a nucleotide variation in SEQ ID NO:1 or a fragment thereof.

In another aspect, a method of detecting the absence or presence of an amino acid variation at P1104, P1105, or W1067 of TYK2 in a biological sample is provided, the method comprising (a) contacting nucleic acid derived from the biological sample with an allele-specific oligonucleotide that is specific for a nucleotide variation under conditions suitable for hybridization of the allele-specific oligonucleotide to the nucleic acid, wherein the nucleotide variation results in the amino acid variation; and (b) detecting the absence or presence of allele-specific hybridization, wherein the presence of allele-specific hybridization indicates the presence of the amino acid variation. In one embodiment, the amino acid variation is an amino acid substitution at P1104. In one such embodiment, the amino acid substitution is P1104A. In another embodiment, the nucleotide variation is a C3651G substitution. In another embodiment, the nucleotide variation comprises a nucleotide variation in SEQ ID NO:1 or a fragment thereof. In another embodiment, an allele-specific oligonucleotide is an allele-specific primer.

In another aspect, a method of amplifying a nucleic acid comprising a nucleotide variation is provided, wherein the nucleotide variation results in an amino acid variation at P1104, P1105, or W1067 of TYK2, the method comprising (a) contacting the nucleic acid with a primer that hybridizes to a sequence 3′ of the nucleotide variation, and (b) extending the primer to generate an amplification product comprising the nucleotide variation. In one embodiment, the amino acid variation is an amino acid substitution at P1104. In one such embodiment, the amino acid substitution is P1104A. In another embodiment, the nucleotide variation is a C3651G substitution. In another embodiment, the nucleic acid comprises a nucleotide variation in SEQ ID NO:1 or a fragment thereof. In another embodiment, the method further comprises contacting the amplification product with a second primer that hybridizes to a sequence 3′ of the nucleotide variation, and extending the second primer to generate a second amplification product. In one such embodiment, the method further comprises amplifying the amplification product and second amplification product, e.g., by PCR.

In another aspect, a method of assessing the activity of a TYK2 is provided, the method comprising (a) determining the activity of a TYK2 comprising an amino acid variation at P1104, P1105, or W1067; and (b) comparing the activity of the TYK2 with the activity of a wild-type TYK2. In one embodiment, the activity is kinase activity. In one embodiment, the amino acid variation is an amino acid substitution at P1104. In one such embodiment, the amino acid substitution is P1104A.

In another aspect, a method of identifying an agent for the treatment of a tumor is provided, the method comprising (a) contacting a TYK2 with a test agent, wherein the TYK2 comprises an amino acid variation at P1104, P1105, or W1067; and (b) assessing the activity of the TYK2 in the presence of the test agent with the activity of the TYK2 in the absence of the test agent, wherein a decrease in the activity of the TYK2 in the presence of the test agent indicates that the test agent is an agent for the treatment of a tumor. In one embodiment, the amino acid variation is an amino acid substitution at P104. In one such embodiment, the amino acid substitution is P1104A. In another embodiment, the tumor is a breast, colon or stomach tumor.

In another aspect, a method of inhibiting the proliferation of a tumor cell is provided, wherein the tumor cell comprises a TYK2 having an amino acid variation in the catalytic kinase domain, the method comprising exposing the tumor cell to an antagonist of TYK2. In one embodiment, the amino acid variation occurs at P1104, P1105, or W1067. In another embodiment, the amino acid variation is an amino acid substitution at P1104. In one such embodiment, the amino acid substitution is P1104A. In another embodiment, the tumor cell is a breast, colon, or stomach tumor cell. In another embodiment, the antagonist of TYK2 is a small molecule antagonist. In another embodiment, the antagonist of TYK2 is an antagonist antibody. In another embodiment, the method further comprises exposing the tumor cell to a cytotoxic agent, chemotherapeutic agent, or growth inhibitory agent.

A variety of kinase antagonists are known in the art. Such kinase antagonists include, but are not limited to antagonist antibodies and small molecule antagonists, e.g., 3-[2,4-dimethylpyrrol-5-yl)methylidene]-indolin-2-one (“SU5416”); imatinib (Gleeve®), a 2-phenylaminopyrimidine; 1-tert-butyl-3-[6-(3,5-dimethoxy-phenyl)-2-(4-diethylamino-butylamino)-pyrido[2,3-d]pyrimidin-7-yl]-urea (“PD173074”) (see, e.g., Moffa et al. (2004) Mol. Cancer. Res. 2:643-652); and indolinones such as 3-[3-(2-carboxyethyl)-4-methylpyrrol-2-methylidenyl]-2-indolinone (“SU5402”) (see, e.g., Bernard-Pierrot (2004) Oncogene 23:9201-9211).

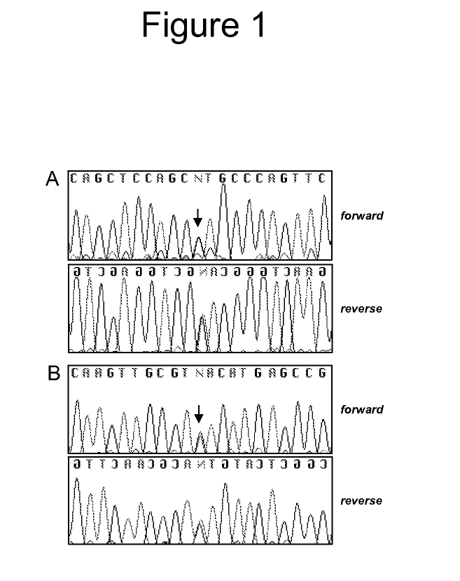

In particular, certain small molecule antagonists of Jak kinases are known in the art. Such small molecule antagonists of Jak kinases include, but are not limited to, ZM449829, ZM39923, indirubin, WHI-P131, PP1, TDZD-8, meta-hydroxybenzylamide indole (2k), tyrophostin 25 (A25) and AG490, as discussed in Luo et al. (2004) Drug Discovery Today 9:268-275 (see FIG. 1 for chemical structures).

In another aspect, a method of treating a tumor comprising a TYK2 having an amino acid variation in the catalytic kinase domain is provided, the method comprising administering to a patient having the tumor an effective amount of a pharmaceutical formulation comprising an antagonist of TYK2. In one embodiment, the amino acid variation occurs at P1104, P1105, or W1067. In another embodiment, the amino acid variation is an amino acid substitution at P1104. In one such embodiment, the amino acid substitution is P1104A. In another embodiment, the tumor is a breast, colon, or stomach tumor. In another embodiment, the antagonist of TYK2 is a small molecule antagonist. In another embodiment, the antagonist of TYK2 is an antagonist antibody. In another embodiment, the method further comprises administering to the patient a cytotoxic agent, chemotherapeutic agent, or growth inhibitory agent.

B. MAST2 Variations

In another example, a cancer-associated mutation in the human microtubule-associated serine/threonine kinase 2 (MAST2) gene has been identified. That mutation results in a V1304M substitution in MAST2.

1. Compositions

In one aspect, an isolated polynucleotide comprising at least a fragment of a MAST2 polynucleotide is provided, wherein the fragment comprises a nucleotide variation that results in an amino acid variation at V1304 of MAST2. In one embodiment, the amino acid variation is a V1304M substitution. The amino acid positions designated herein refer to amino acid positions in the human MAST2 amino acid sequence shown in SEQ ID NO:4. The designated amino acid positions also refer to the corresponding amino acid positions in fragments or variants of SEQ ID NO:4 (e.g., allelic variants or splice variants), which can be routinely identified using sequence alignments or similar techniques. In another embodiment, a MAST2 polynucleotide or fragment thereof comprises a nucleotide variation in the sequence of SEQ ID NO:3 or a fragment of SEQ ID NO:3. In one embodiment, a fragment of SEQ ID NO:3 is the coding region of SEQ ID NO:3 from nucleotides 258-5651.

In another embodiment, a fragment of a MAST2 polynucleotide is at least about 10 nucleotides in length, alternatively at least about 15 nucleotides in length, alternatively at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length, alternatively at least about 1000 nucleotides in length, and alternatively about the length of the full-length coding sequence. In this context the term “about” means the referenced nucleotide sequence length plus or minus 10% of that referenced length. In another embodiment, a MAST2 polynucleotide comprises the nucleic acid sequence of SEQ ID NO:3 or a coding region thereof. In another embodiment, the complement of any of the above polynucleotides is provided. In another embodiment, a MAST2 encoded by the any of the above polynucleotides is provided.

In one embodiment, an isolated polynucleotide provided herein is detectably labeled, e.g., with a radioisotope, a fluorescent agent, or a chromogenic agent. In another embodiment, an isolated polynucleotide is a primer. In another embodiment, an isolated polynucleotide is an oligonucleotide, e.g., an allele-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Oligonucleotides as provided herein are useful, e.g., as hybridization probes for the detection of tumor-associated variations.

In another aspect, an allele-specific oligonucleotide is provided that hybridizes to a region of a MAST2 polynucleotide comprising a nucleotide variation (e.g., a substitution) that results in an amino acid variation at V1304 of MAST2. The allele-specific oligonucleotide, when hybridized to the region of the MAST2 polynucleotide, comprises a nucleotide that base pairs with the nucleotide variation. In one embodiment, the amino acid variation is a V1304M substitution. In another embodiment, the complement of an allele-specific oligonucleotide is provided. In another embodiment, a microarray comprises an allele-specific oligonucleotide or its complement. In another embodiment, an allele-specific oligonucleotide or its complement is an allele-specific primer.

An allele-specific oligonucleotide can be used in conjunction with a control oligonucleotide that is identical to the allele-specific oligonucleotide, except that the nucleotide that specifically base pairs with the nucleotide variation is replaced with a nucleotide that specifically base pairs with the corresponding nucleotide present in the wild type MAST2 polynucleotide. Such oligonucleotides may be used in competitive binding assays under hybridization conditions that allow the oligonucleotides to distinguish between a MAST2 polynucleotide comprising a nucleotide variation and a MAST2 polynucleotide comprising the corresponding wild type nucleotide. Using routine methods based on, e.g., the length and base composition of the oligonucleotides, one skilled in the art can arrive at suitable hybridization conditions under which (a) an allele-specific oligonucleotide will preferentially bind to a MAST2 polynucleotide comprising a nucleotide variation relative to a wild type MAST2 polynucleotide, and (b) the control oligonucleotide will preferentially bind to a wild type MAST2 polynucleotide relative to a MAST2 polynucleotide comprising a nucleotide variation. Exemplary conditions include conditions of high stringency, e.g., hybridization conditions of 5× standard saline phosphate EDTA (SSPE) and 0.5% NaDodSO₄ (SDS) at 55° C., followed by washing with 2×SSPE and 0.1% SDS at 55° C. or room temperature.

In another aspect, a binding agent is provided that preferentially binds to a MAST2 comprising an amino acid variation at V1304, relative to a MAST2 encoded by a wild-type MAST2 polynucleotide. In one embodiment, the amino acid variation is a V1304M substituion. In another embodiment, the binding agent is an antibody. In another embodiment, the binding agent is an oligopeptide that binds MAST2 comprising an amino acid variation at V1304.

In another aspect, diagnostic kits are provided. In one embodiment, a kit comprises any of the foregoing polynucleotides and an enzyme. In one embodiment, the enzyme is at least one enzyme selected from a nuclease, a ligase, and a polymerase.

2. Methods

In one aspect, a method of detecting the presence of a tumor in a biological sample derived from a patient is provided, the method comprising detecting an amino acid variation at V1304 of MAST2 in the biological sample. Detecting an amino acid variation encompasses detecting the amino acid variation directly (e.g., with an antibody that preferentially recognizes a polypeptide comprising the amino acid variation relative to the corresponding wild-type polypeptide, or vice versa) or indirectly (e.g., by detecting a nucleotide variation in a polynucleotide that encodes a polypeptide comprising the amino acid variation). In one embodiment, the amino acid variation is a V1304M substitution. In another embodiment, the amino acid variation is detected by detecting a nucleotide variation in the biological sample that results in the amino acid variation. In one such embodiment, the nucleotide variation comprises a nucleotide variation in SEQ ID NO:3 or a coding region thereof. In another embodiment, the biological sample is a biopsy (e.g., a tissue sample containing cells suspected of being cancerous).

In another aspect, a method of diagnosing a tumor in a patient is provided, the method comprising detecting the presence of an amino acid variation at V1304 of MAST2 in a biological sample derived from the patient. In one embodiment, the amino acid variation is a V1304M substitution. In another embodiment, the biological sample comprises or is suspected of comprising tumor cells. In another embodiment, the amino acid variation is detected by detecting a nucleotide variation in the biological sample that results in the amino acid variation. In one such embodiment, the nucleotide variation comprises a nucleotide variation in SEQ ID NO:3 or a coding region thereof.

In another aspect, a method is provided for determining whether a tumor in a patient will respond to a therapeutic agent that targets a MAST2 or MAST2 polynucleotide, the method comprising detecting the absence or presence of an amino acid variation at V1304 of MAST2 in a biological sample derived from the patient, wherein the presence of the amino acid variation indicates that the tumor will respond to the therapeutic agent. In one embodiment, the amino acid variation is a V1304M substitution. In another embodiment, the amino acid variation is detected by detecting a nucleotide variation in a MAST2 polynucleotide derived from the biological sample, wherein the nucleotide variation results in the amino acid variation. In one such embodiment, the nucleotide variation comprises a nucleotide variation in SEQ ID NO:3 or a coding region thereof.

In another aspect, a method of detecting the absence or presence of an amino acid variation at V1304 of MAST2 in a biological sample is provided, the method comprising (a) contacting nucleic acid derived from the biological sample with any of the above-described polynucleotides under conditions suitable for formation of a hybridization complex between the polynucleotide and the nucleic acid; and (b) detecting whether at least one nucleotide in the nucleic acid specifically base pairs with the polynucleotide in the hybridization complex, wherein the at least one nucleotide is suspected of comprising a nucleotide variation that results in the amino acid variation, and wherein the presence of specific base pairing indicates the presence of the amino acid variation. In one embodiment, the amino acid variation is a V1304M substitution. In one such embodiment, the nucleotide variation comprises a nucleotide variation in SEQ ID NO:3 or a fragment thereof.

In another aspect, a method of detecting the absence or presence of an amino acid variation at V1304 of MAST2 in a biological sample is provided, the method comprising (a) contacting nucleic acid derived from the biological sample with an allele-specific oligonucleotide that is specific for a nucleotide variation under conditions suitable for hybridization of the allele-specific oligonucleotide to the nucleic acid, wherein the nucleotide variation results in the amino acid variation; and (b) detecting the absence or presence of allele-specific hybridization, wherein the presence of allele-specific hybridization indicates the presence of the amino acid variation. In one embodiment, the amino acid variation is a V1304M substitution. In another embodiment, the nucleotide variation comprises a nucleotide variation in SEQ ID NO:3 or a fragment thereof. In another embodiment, an allele-specific oligonucleotide is an allele-specific primer.

In another aspect, a method of amplifying a nucleic acid comprising a nucleotide variation is provided, wherein the nucleotide variation results in an amino acid variation at V1304 of MAST2, the method comprising (a) contacting the nucleic acid with a primer that hybridizes to a sequence 3′ of the nucleotide variation, and (b) extending the primer to generate an amplification product comprising the nucleotide variation. In one embodiment, the amino acid variation is a V1304M substitution. In another embodiment, the nucleic acid comprises a nucleotide variation in SEQ ID NO:3 or a fragment thereof. In another embodiment, the method further comprises contacting the amplification product with a second primer that hybridizes to a sequence 3′ of the nucleotide variation, and extending the second primer to generate a second amplification product. In one such embodiment, the method further comprises amplifying the amplification product and second amplification product, e.g., by PCR.

In another aspect, a method of assessing the activity of a MAST2 is provided, the method comprising (a) determining the activity of a MAST2 comprising an amino acid variation at V1304; and (b) comparing the activity of the MAST2 with the activity of a wild-type MAST2. In one embodiment, the activity is kinase activity. In one embodiment, the amino acid variation is a V1304M substitution.

In another aspect, a method of identifying an agent for the treatment of a tumor is provided, the method comprising (a) contacting a MAST2 with a test agent, wherein the MAST2 comprises an amino acid variation at V1304; and (b) assessing the activity of the MAST2 in the presence of the test agent with the activity of the MAST2 in the absence of the test agent, wherein a decrease in the activity of the MAST2 in the presence of the test agent indicates that the test agent is an agent for the treatment of a tumor. In one embodiment, the amino acid variation is a V1304M substitution.

In another aspect, a method of inhibiting the proliferation of a tumor cell is provided, wherein the tumor cell comprises a MAST2 having an amino acid variation at V1304, the method comprising exposing the tumor cell to an antagonist of MAST2. In one embodiment, the amino acid variation is a V1304M substitution. In another embodiment, the antagonist of MAST2 is a small molecule antagonist. In another embodiment, the antagonist of MAST2 is an antagonist antibody. In another embodiment, the method further comprises exposing the tumor cell to a cytotoxic agent, chemotherapeutic agent, or growth inhibitory agent.

A variety of kinase antagonists are known in the art. Such kinase antagonists include, but are not limited to, antagonist antibodies and small molecule antagonists, e.g., 3-[2,4-dimethylpyrrol-5-yl)methylidene]-indolin-2-one (“SU5416”); imatinib (Gleevec®), a 2-phenylaminopyrimidine; 1-tert-butyl-3-[6-(3,5-dimethoxy-phenyl)-2-(4-diethylamino-butylamino)-pyrido[2,3-d]pyrimidin-7-yl]-urea (“PD173074”) (see, e.g., Moffa et al. (2004) Mol. Cancer. Res. 2:643-652); and indolinones such as 3-[3-(2-carboxyethyl)-4-methylpyrrol-2-methylidenyl]-2-indolinone (“SU5402”) (see, e.g., Bernard-Pierrot (2004) Oncogene 23:9201-9211).

In another aspect, a method of treating a tumor comprising a MAST2 having an amino acid variation at V1304 is provided, the method comprising administering to a patient having the tumor an effective amount of a pharmaceutical formulation comprising an antagonist of MAST2. In one embodiment, the amino acid variation is a V1304M substitution. In another embodiment, the antagonist of MAST2 is a small molecule antagonist. In another embodiment, the antagonist of MAST2 is an antagonist antibody. In another embodiment, the method further comprises administering to the patient a cytotoxic agent, chemotherapeutic agent, or growth inhibitory agent.

C. RIOK2 Variations

In another example, a cancer-associated mutation in the human serine-threonine protein kinase RIO2 (RIOK2) gene has been identified. That mutation results in a Y11H substitution in RIOK2.

1. Compositions

In one aspect, an isolated polynucleotide comprising at least a fragment of a RIOK2 polynucleotide is provided, wherein the fragment comprises a nucleotide variation that results in an amino acid variation at Y11 of RIOK2. In one embodiment, the amino acid variation is a Y11H substitution. The amino acid positions designated herein refer to amino acid positions in the human RIOK2 amino acid sequence shown in SEQ ID NO:6. The designated amino acid positions also refer to the corresponding amino acid positions in fragments or variants of SEQ ID NO:6 (e.g., allelic variants or splice variants), which can be routinely identified using sequence alignments or similar techniques. In another embodiment, a RIOK2 polynucleotide or fragment thereof comprises a nucleotide variation in the sequence of SEQ ID NO:5 or a fragment of SEQ ID NO:5. In one embodiment, a fragment of SEQ ID NO:5 is the coding region of SEQ ID NO:5 from nucleotides 50-1708.

In one embodiment, a fragment of a RIOK2 polynucleotide is at least about 10 nucleotides in length, alternatively at least about 15 nucleotides in length, alternatively at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length, alternatively at least about 1000 nucleotides in length, and alternatively about the length of the full-length coding sequence. In this context the term “about” means the referenced nucleotide sequence length plus or minus 10% of that referenced length. In another embodiment, a RIOK2 polynucleotide comprises the nucleic acid sequence of SEQ ID NO:5 or a coding region thereof. In another embodiment, the complement of any of the above polynucleotides is provided. In another embodiment, a RIOK2 encoded by the any of the above polynucleotides is provided.

In one embodiment, an isolated polynucleotide provided herein is detectably labeled, e.g., with a radioisotope, a fluorescent agent, or a chromogenic agent. In another embodiment, an isolated polynucleotide is a primer. In another embodiment, an isolated polynucleotide is an oligonucleotide, e.g., an allele-specific oligonucleotide. In another embodiment, an oligonucleotide may be, for example, from 7-60 nucleotides in length, 9-45 nucleotides in length, 15-30 nucleotides in length, or 18-25 nucleotides in length. In another embodiment, an oligonucleotide may be, e.g., PNA, morpholino-phosphoramidates, LNA, or 2′-alkoxyalkoxy. Oligonucleotides as provided herein are useful, e.g., as hybridization probes for the detection of tumor-associated variations.

In another aspect, an allele-specific oligonucleotide is provided that hybridizes to a region of a RIOK2 polynucleotide comprising a nucleotide variation (e.g., a substitution) that results in an amino acid variation at Y11 of RIOK2. The allele-specific oligonucleotide, when hybridized to the region of the RIOK2 polynucleotide, comprises a nucleotide that base pairs with the nucleotide variation. In one embodiment, the amino acid variation is a Y11H substitution. In another embodiment, the complement of an allele-specific oligonucleotide is provided. In another embodiment, a microarray comprises an allele-specific oligonucleotide or its complement. In another embodiment, an allele-specific oligonucleotide or its complement is an allele-specific primer.

An allele-specific oligonucleotide can be used in conjunction with a control oligonucleotide that is identical to the allele-specific oligonucleotide, except that the nucleotide that specifically base pairs with the nucleotide variation is replaced with a nucleotide that specifically base pairs with the corresponding nucleotide present in the wild type RIOK2 polynucleotide. Such oligonucleotides may be used in competitive binding assays under hybridization conditions that allow the oligonucleotides to distinguish between a RIOK2 polynucleotide comprising a nucleotide variation and a RIOK2 polynucleotide comprising the corresponding wild type nucleotide. Using routine methods based on, e.g., the length and base composition of the oligonucleotides, one skilled in the art can arrive at suitable hybridization conditions under which (a) an allele-specific oligonucleotide will preferentially bind to a RIOK2 polynucleotide comprising a nucleotide variation relative to a wild type RIOK2 polynucleotide, and (b) the control oligonucleotide will preferentially bind to a wild type RIOK2 polynucleotide relative to a RIOK2 polynucleotide comprising a nucleotide variation. Exemplary conditions include conditions of high stringency, e.g., hybridization conditions of 5× standard saline phosphate EDTA (SSPE) and 0.5% NaDodSO₄ (SDS) at 55° C., followed by washing with 2×SSPE and 0.1% SDS at 55° C. or room temperature.

In another aspect, a binding agent is provided that preferentially binds to a RIOK2 comprising an amino acid variation at Y11, relative to a RIOK2 encoded by a wild-type RIOK2 polynucleotide. In one embodiment, the amino acid variation is a Y11H substituion. In another embodiment, the binding agent is an antibody. In another embodiment, the binding agent is an oligopeptide that binds RIOK2 comprising an amino acid variation at Y11.

In another aspect, diagnostic kits are provided. In one embodiment, a kit comprises any of the foregoing polynucleotides and an enzyme. In one embodiment, the enzyme is at least one enzyme selected from a nuclease, a ligase, and a polymerase.

2. Methods

In one aspect, a method of detecting the presence of a tumor in a biological sample derived from a patient is provided, the method comprising detecting an amino acid variation at Y11 of RIOK2 in the biological sample. Detecting an amino acid variation encompasses detecting the amino acid variation directly (e.g., with an antibody that preferentially recognizes a polypeptide comprising the amino acid variation relative to the corresponding wild-type polypeptide, or vice versa) or indirectly (e.g., by detecting a nucleotide variation in a polynucleotide that encodes a polypeptide comprising the amino acid variation). In one embodiment, the amino acid variation is a Y11H substitution. In another embodiment, the amino acid variation is detected by detecting a nucleotide variation in the biological sample that results in the amino acid variation. In one such embodiment, the nucleotide variation comprises a nucleotide variation in SEQ ID NO:5 or a coding region thereof. In another embodiment, the biological sample is a biopsy (e.g., a tissue sample containing cells suspected of being cancerous).

In another aspect, a method of diagnosing a tumor in a patient is provided, the method comprising detecting the presence of an amino acid variation at Y11 of RIOK2 in a biological sample derived from the patient. In one embodiment, the amino acid variation is a Y11H substitution. In another embodiment, the biological sample comprises or is suspected of comprising tumor cells. In another embodiment, the amino acid variation is detected by detecting a nucleotide variation in the biological sample that results in the amino acid variation. In one such embodiment, the nucleotide variation comprises a nucleotide variation in SEQ ID NO:5 or a coding region thereof.

In another aspect, a method is provided for determining whether a tumor in a patient will respond to a therapeutic agent that targets a RIOK2 or RIOK2 polynucleotide, the method comprising detecting the absence or presence of an amino acid variation at Y11 of RIOK2 in a biological sample derived from the patient, wherein the presence of the amino acid variation indicates that the tumor will respond to the therapeutic agent. In one embodiment, the amino acid variation is a Y11H substitution. In another embodiment, the amino acid variation is detected by detecting a nucleotide variation in a RIOK2 polynucleotide derived from the biological sample, wherein the nucleotide variation results in the amino acid variation. In one such embodiment, the nucleotide variation in the RIOK2 polynucleotide comprises a nucleotide variation in SEQ ID NO:5 or a coding region thereof.

In another aspect, a method of detecting the absence or presence of an amino acid variation at Y11 of RIOK2 in a biological sample is provided, the method comprising (a) contacting nucleic acid derived from the biological sample with any of the above-described polynucleotides under conditions suitable for formation of a hybridization complex between the polynucleotide and the nucleic acid; and (b) detecting whether at least one nucleotide in the nucleic acid specifically base pairs with the polynucleotide in the hybridization complex, wherein the at least one nucleotide is suspected of comprising a nucleotide variation that results in the amino acid variation, and wherein the presence of specific base pairing indicates the presence of the amino acid variation. In one embodiment, the amino acid variation is a Y11H substitution. In another embodiment, the nucleotide variation comprises a nucleotide variation in SEQ ID NO:5 or a fragment thereof.

In another aspect, a method of detecting the absence or presence of an amino acid variation at Y11 of RIOK2 in a biological sample is provided, the method comprising (a) contacting nucleic acid derived from the biological sample with an allele-specific oligonucleotide that is specific for a nucleotide variation under conditions suitable for hybridization of the allele-specific oligonucleotide to the nucleic acid, wherein the nucleotide variation results in the amino acid variation; and (b) detecting the absence or presence of allele-specific hybridization, wherein the presence of allele-specific hybridization indicates the presence of the amino acid variation. In one embodiment, the amino acid variation is a Y11H substitution. In another embodiment, the nucleotide variation comprises a nucleotide variation in SEQ ID NO:5 or a fragment thereof. In another embodiment, an allele-specific oligonucleotide is an allele-specific primer.

In another aspect, a method of amplifying a nucleic acid comprising a nucleotide variation is provided, wherein the nucleotide variation results in an amino acid variation at Y11 of RIOK2, the method comprising (a) contacting the nucleic acid with a primer that hybridizes to a sequence 3′ of the nucleotide variation, and (b) extending the primer to generate an amplification product comprising the nucleotide variation. In one embodiment, the amino acid variation is a Y11H substitution. In another embodiment, the nucleic acid comprises a nucleotide variation in SEQ ID NO:5 or a fragment thereof. In another embodiment, the method further comprises contacting the amplification product with a second primer that hybridizes to a sequence 3′ of the nucleotide variation, and extending the second primer to generate a second amplification product. In one such embodiment, the method further comprises amplifying the amplification product and second amplification product, e.g., by PCR.

In another aspect, a method of assessing the activity of a RIOK2 is provided, the method comprising (a) determining the activity of a RIOK2 comprising an amino acid variation at Y11; and (b) comparing the activity of the RIOK2 with the activity of a wild-type RIOK2. In one embodiment, the activity is kinase activity. In one embodiment, the amino acid variation is a Y11H substitution.

In another aspect, a method of identifying an agent for the treatment of a tumor is provided, the method comprising (a) contacting a RIOK2 with a test agent, wherein the RIOK2 comprises an amino acid variation at Y11; and (b) assessing the activity of the RIOK2 in the presence of the test agent with the activity of the RIOK2 in the absence of the test agent, wherein a decrease in the activity of the RIOK2 in the presence of the test agent indicates that the test agent is an agent for the treatment of a tumor. In one embodiment, the amino acid variation is a Y11H substitution.

In another aspect, a method of inhibiting the proliferation of a tumor cell is provided, wherein the tumor cell comprises a RIOK2 having an amino acid variation at Y11, the method comprising exposing the tumor cell to an antagonist of RIOK2. In one embodiment, the amino acid variation is a Y11H substitution. In another embodiment, the antagonist of RIOK2 is a small molecule antagonist. In another embodiment, the antagonist of RIOK2 is an antagonist antibody. In another embodiment, the method further comprises exposing the tumor cell to a cytotoxic agent, chemotherapeutic agent, or growth inhibitory agent.

A variety of kinase antagonists are known in the art. Such kinase antagonists include, but are not limited to antagonist antibodies and small molecule antagonists, e.g., 3-[2,4-dimethylpyrrol-5-yl)methylidene]-indolin-2-one (“SU5416”); imatinib (Gleevec®), a 2-phenylaminopyrimidine; 1-tert-butyl-3-[6-(3,5-dimethoxy-phenyl)-2-(4-diethylamino-butylamino)-pyrido[2,3-d]pyrimidin-7-yl]-urea (“PD173074”) (see, e.g., Moffa et al. (2004) Mol. Cancer. Res. 2:643-652); and indolinones such as 3-[3-(2-carboxyethyl)-4-methylpyrrol-2-methylidenyl]-2-indolinone (“SU5402”) (see, e.g., Bernard-Pierrot (2004) Oncogene 23:9201-9211).

In another aspect, a method of treating a tumor comprising a RIOK2 having an amino acid variation at Y11 is provided, the method comprising administering to a patient having the tumor an effective amount of a pharmaceutical formulation comprising an antagonist of RIOK2. In one embodiment, the amino acid variation is a Y11H substitution. In another embodiment, the antagonist of RIOK2 is a small molecule antagonist. In another embodiment, the antagonist of RIOK2 is an antagonist antibody. In another embodiment, the method further comprises administering to the patient a cytotoxic agent, chemotherapeutic agent, or growth inhibitory agent.

D. General Techniques

Nucleic acid, according to any of the above methods, may be genomic DNA; RNA transcribed from genomic DNA; or cDNA generated from RNA. Nucleic acid may be derived from a vertebrate, e.g., a mammal. A nucleic acid is said to be “derived from” a particular source if it is obtained directly from that source or if it is a copy of a nucleic acid found in that source.

Nucleic acid includes copies of the nucleic acid, e.g., copies that result from amplification. Amplification may be desirable in certain instances, e.g., in order to obtain a desired amount of material for detecting variations. For example, a TYK2 polynucleotide or portion thereof may be amplified from nucleic acid material. The amplicons may then be subjected to a variation detection method, such as those described below, to determine whether a variation is present in the amplicon.

Variations may be detected by certain methods known to those skilled in the art. Such methods include, but are not limited to, DNA sequencing; primer extension assays, including allele-specific nucleotide incorporation assays and allele-specific primer extension assays (e.g., allele-specific PCR, allele-specific ligation chain reaction (LCR), and gap-LCR); allele-specific oligonucleotide hybridization assays (e.g., oligonucleotide ligation assays); cleavage protection assays in which protection from cleavage agents is used to detect mismatched bases in nucleic acid duplexes; analysis of MutS protein binding; electrophoretic analysis comparing the mobility of variant and wild type nucleic acid molecules; denaturing-gradient gel electrophoresis (DGGE, as in, e.g., Myers et al. (1985) Nature 313:495; analysis of RNase cleavage at mismatched base pairs; analysis of chemical or enzymatic cleavage of heteroduplex DNA; mass spectrometry (e.g., MALDI-TOF); genetic bit analysis (GBA); 5′ nuclease assays (e.g., TaqMan®); and assays employing molecular beacons. Certain of these methods are discussed in further detail below.

Detection of variations in target nucleic acids may be accomplished by molecular cloning and sequencing of the target nucleic acids using techniques well known in the art. Alternatively, amplification techniques such as the polymerase chain reaction (PCR) can be used to amplify target nucleic acid sequences directly from a genomic DNA preparation from the tumor tissue. The nucleic acid sequence of the amplified sequences can then be determined and variations identified therefrom. Amplification techniques are well known in the art, e.g., polymerase chain reaction is described in Saiki et al., Science 239:487, 1988; U.S. Pat. Nos. 4,683,203 and 4,683,195.

The ligase chain reaction, which is known in the art, can also be used to amplify target nucleic acid sequences. See, e.g., Wu et al., Genomics 4:560-569 (1989). In addition, a technique known as allele-specific PCR can also be used to detect variations (e.g., substitutions). See, e.g., Ruano and Kidd (1989) Nucleic Acids Research 17:8392; McClay et al. (2002) Analytical Biochem. 301:200-206. In certain embodiments of this technique, an allele-specific primer is used wherein the 3′ terminal nucleotide of the primer is complementary to (i.e., capable of specifically base-pairing with) a particular variation in the target nucleic acid. If the particular variation is not present, an amplification product is not observed. Amplification Refractory Mutation System (ARMS) can also be used to detect variations (e.g., substitutions). ARMS is described, e.g., in European Patent Application Publication No. 0332435, and in Newton et al., Nucleic Acids Research, 17:7, 1989.

Other methods useful for detecting variations (e.g., substitutions) include, but are not limited to, (1) allele-specific nucleotide incorporation assays, such as single base extension assays (see, e.g., Chen et al. (2000) Genome Res. 10:549-557; Fan et al. (2000)Genome Res. 10:853-860; Pastinen et al. (1997) Genome Res. 7:606-614; and Ye et al. (2001) Hum. Mut. 17:305-316); (2) allele-specific primer extension assays (see, e.g., Ye et al. (2001) Hum. Mut. 17:305-316; and Shen et al. Genetic Engineering News, vol. 23, Mar. 15, 2003), including allele-specific PCR; (3) 5′nuclease assays (see, e.g., De La Vega et al. (2002) BioTechniques 32:S48-S54 (describing the TaqMan® assay); Ranade et al. (2001) Genome Res. 11:1262-1268; and Shi (2001) Clin. Chem. 47:164-172); (4) assays employing molecular beacons (see, e.g., Tyagi et al. (1998) Nature Biotech. 16:49-53; and Mhlanga et al. (2001) Methods 25:463-71); and (5) oligonucleotide ligation assays (see, e.g., Grossman et al. (1994) Nuc. Acids Res. 22:4527-4534; patent application Publication No. US 2003/0119004 A1; PCT International Publication No. WO 01/92579 A2; and U.S. Pat. No. 6,027,889).

Variations may also be detected by mismatch detection methods. Mismatches are hybridized nucleic acid duplexes which are not 100% complementary. The lack of total complementarity may be due to deletions, insertions, inversions, or substitutions. While these techniques can be less sensitive than sequencing, they are simpler to perform on a large number of tissue samples. An example of a mismatch cleavage technique is the RNase protection method, which is described in detail in Winter et al., Proc. Natl. Acad. Sci. USA, 82:7575, 1985, and Myers et al., Science 230:1242, 1985. For example, a method of the invention may involve the use of a labeled riboprobe which is complementary to the human wild-type target nucleic acid. The riboprobe and target nucleic acid derived from the tissue sample are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the target nucleic acid, but can a portion of the target nucleic acid, provided it encompasses the position suspected of having a variation.

In a similar manner, DNA probes can be used to detect mismatches, for example through enzymatic or chemical cleavage. See, e.g., Cotton et al., Proc. Natl. Acad. Sci. USA, 85:4397, 1988; and Shenk et al., Proc. Natl. Acad. Sci. USA, 72:989, 1975. Alternatively, mismatches can be detected by shifts in the electrophoretic mobility of mismatched duplexes relative to matched duplexes. See, e.g., Cariello, Human Genetics, 42:726, 1988. With either riboprobes or DNA probes, the target nucleic acid suspected of comprising a variation may be amplified before hybridization. Changes in target nucleic acid can also be detected using Southern hybridization, especially if the changes are gross rearrangements, such as deletions and insertions.

Restriction fragment length polymorphism (RFLP) probes for the target nucleic acid or surrounding marker genes can be used to detect variations, e.g., insertions or deletions. Insertions and deletions can also be detected by cloning, sequencing and amplification of a target nucleic acid. Single stranded conformation polymorphism (SSCP) analysis can also be used to detect base change variants of an allele. See, e.g. Orita et al., Proc. Natl. Acad. Sci. USA 86:2766-2770, 1989, and Genomics, 5:874-879, 1989.

Amino acid variations may be detected directly, for example, using antibodies that preferentially recognize a polypeptide comprising an amino acid variation relative to the corresponding wild type polypeptide.

The invention also provides a variety of compositions suitable for use in performing methods of the invention. For example, the invention provides arrays that can be used in such methods. In one embodiment, an array of the invention comprises individual or collections of nucleic acid molecules useful for detecting variations of the invention. For instance, an array of the invention may comprise a series of discretely placed individual allele-specific oligonucleotides or sets of allele-specific oligonucleotides. Several techniques are well-known in the art for attaching nucleic acids to a solid substrate such as a glass slide. One method is to incorporate modified bases or analogs that contain a reactive moiety that is capable of attachment to a solid substrate, such as an amine group, a derivative of an amine group, or another group with a positive charge, into nucleic acid molecules that are synthesized. The synthesized product is then contacted with a solid substrate, such as a glass slide coated with an aldehyde or other reactive group. The aldehyde or other reactive group will form a covalent link with the reactive moiety on the amplified product, which will become covalently attached to the glass slide. Other methods, such as those using amino propryl silican surface chemistry are also known in the art.

A biological sample, according to any of the above methods, may be obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Variations in target nucleic acids (or encoded polypeptides) may be detected from a tumor sample or from other body samples such as urine, sputum or serum. (Cancer cells are sloughed off from tumors and appear in such body samples.) By screening such body samples, a simple early diagnosis can be achieved for diseases such as cancer. In addition, the progress of therapy can be monitored more easily by testing such body samples for variations in target nucleic acids (or encoded polypeptides). Additionally, methods for enriching a tissue preparation for tumor cells are known in the art. For example, the tissue may be isolated from paraffin or cryostat sections. Cancer cells may also be separated from normal cells by flow cytometry or laser capture microdissection.

### III. Examples

### A. Identification of Cancer-Associated Variations

Cancer-associated variations were identified in ESTs using a computational classifier, or “random forest” (RF) classifier, which was constructed using information from SIFT, the Pfam-based LogR.E-value, and Gene Ontology Similarity Score metrics as applied to a training set of known cancer-associated variations. The RF classifier is capable of distinguishing cancer-associated variations from common polymorphisms. See Kaminker et al. (2007) Cancer Research. The RF classifier was used to analyze a collection of 2600 candidate variations that were specifically present in ESTs from cancer libraries. Out of the 2600 candidate variations, the RF classifier identified 494 (19%) as being cancer-associated. The 494 cancer-associated variations included a number of known cancer-associated mutations such as the C135F change in p53.

To confirm that the identified cancer-associated variations were not due to EST sequencing artifacts, eight variations were validated by determining whether the variations were present in corresponding genomic sequences. Two of the variations, a V1304M change in MAST2 and a Y11H change in RIOK2, were also identified in genomic DNA from their respective EST tissue sources. (See FIG. 1.)

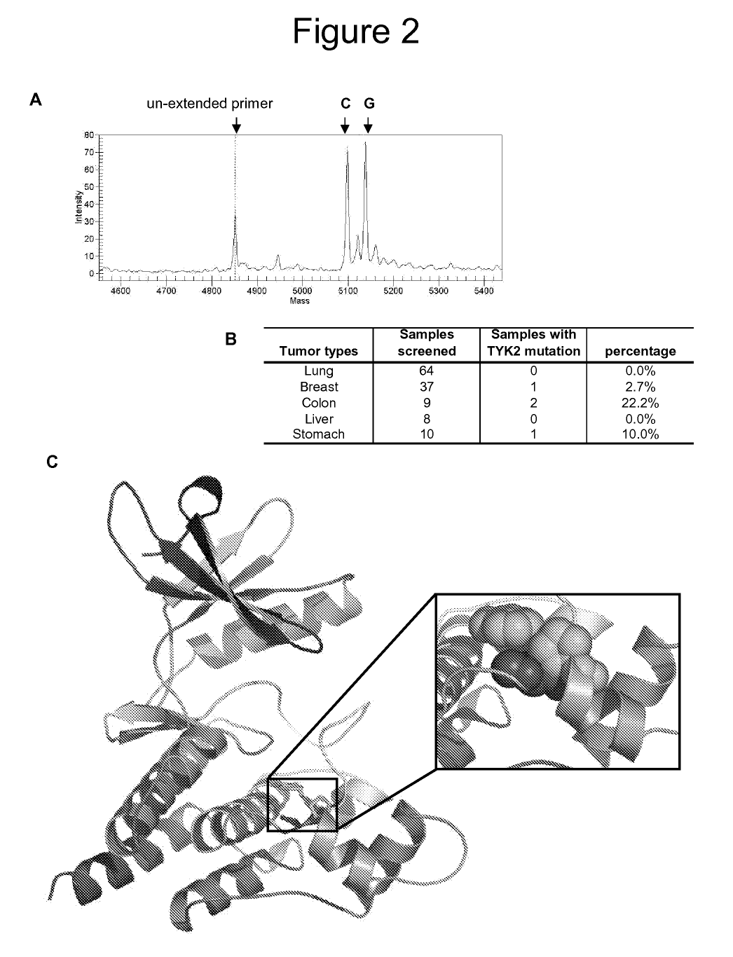

To identify cancer-associated mutations that are present in unrelated tumor DNA samples, mass spectrometry genotyping was performed for 65 predicted cancer-associated mutations over a collection of 128 tumor tissue samples that were independent of any EST libraries used in the screen. From this analysis, a novel variation in the kinase domain of the TYK2 gene was identified in 4 independent tumor tissues: one breast tumor, two colon tumor, and one stomach tumor. (See FIGS. 2A and 2B.) This variation, P1104A, results from a C3651G mutation. The mutation was classified as a germline change as it was also identified in matched normal tissue samples.

FIG. 2C shows the predicted structure of the TYK2 kinase domain. Proline 1104 is indicated in black in the boxed region, and proline 1105 and tryptophan 1067 are indicated in light grey in the boxed region. As shown in FIG. 2C, P1104 is a conserved residue that lies in the substrate-binding groove in the C-terminal, helical lobe of the TYK2 kinase domain. It is positioned under a key tryptophan residue, W1067, in a ring-stacking interaction that stabilizes the inactive conformation of the activation loop. Mutation of P1104 could thus precipitate an activated catalytic state of TYK2 and lead to an oncogenic phenotype. P1104 also contacts the ring of the neighboring P1105 residue, which helps position the adjacent α-helix. Any disruption of this tightly packed trio of amino acids may affect the catalytic activity of TYK2 by either altering the substrate binding groove or the conformation of the active loop.

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

1. A method of diagnosing a colon or stomach tumor in a patient comprising detecting the presence of an amino acid variation in the catalytic kinase domain of TYK2 in a biological sample derived from the patient.

2. The method of claim 1, wherein the amino acid variation occurs at P1104, P1105, or W1067.

3. The method of claim 2, wherein the amino acid variation is an amino acid substitution at P1104

4. The method of claim 3, wherein the amino acid substitution is P1104A.

5. The method of claim 2, wherein the detecting comprises detecting the presence of a nucleotide variation in a TYK2 polynucleotide derived from the biological sample, wherein the nucleotide variation results in the amino acid variation.

6. The method of claim 5, wherein the nucleotide variation is a C3651G substitution.

7. The method of claim 5, wherein the nucleotide variation in the TYK2 polynucleotide comprises a nucleotide variation in SEQ ID NO:1 or a coding region thereof.

8. The method of claim 1, wherein the biological sample comprises or is suspected of comprising tumor cells.

9. (canceled)

10. A method for determining whether a colon or stomach tumor in a patient will respond to a therapeutic agent that targets a TYK2 or TYK2 polynucleotide, the method comprising detecting the absence or presence of an amino acid variation in the catalytic kinase domain of TYK2 in a biological sample derived from the patient, wherein the presence of the amino acid variation indicates that the colon or stomach tumor will respond to the therapeutic agent.

11. The method of claim 10, wherein the amino acid variation occurs at P1104, P1105, or W1067.

12. The method of claim 11, wherein the amino acid variation is an amino acid substitution at P1104.

13. The method of claim 12, wherein the amino acid substitution is P1104A.

14. (canceled)

15. The method of claim 11, wherein the amino acid variation is detected by detecting a nucleotide variation in a TYK2 polynucleotide derived from the biological sample, wherein the nucleotide variation results in the amino acid variation.

16. The method of claim 15, wherein the nucleotide variation is a C3651G substitution.

17. The method of claim 15, wherein the nucleotide variation in the TYK2 polynucleotide comprises a nucleotide variation in SEQ ID NO:1 or a coding region thereof.

18. A method of inhibiting the proliferation of a tumor cell, wherein the tumor cell comprises a TYK2 having an activating amino acid variation in the catalytic kinase domain, the method comprising exposing the tumor cell to an antagonist of TYK2.

19. The method of claim 18, wherein the amino acid variation occurs at P1104, P1105, or W1067.

20. The method of claim 19, wherein the amino acid variation is an amino acid substitution at P1104.

21. The method of claim 20, wherein the amino acid substitution is P1104A.

22. The method of claim 18, wherein the tumor cell is a breast, colon, or stomach tumor cell.

23. The method of claim 18, wherein the antagonist of TYK2 is a small molecule antagonist.

24. The method of claim 18, wherein the antagonist of TYK2 is an antagonist antibody.

25. The method of claim 18, further comprising exposing the cell to a cytotoxic agent, chemotherapeutic agent, or growth inhibitory agent.

26. A method of treating a tumor comprising a TYK2 having an activating amino acid variation in the catalytic domain, the method comprising administering to a patient having the tumor an effective amount of a pharmaceutical formulation comprising an antagonist of TYK2.

27. The method of claim 26, wherein the amino acid variation occurs at P1104, P1105, or W1067.

28. The method of claim 27, wherein the amino acid variation is an amino acid substitution at P1104.

29. The method of claim 28, wherein the amino acid substitution is P1104A.

30. The method of claim 26, wherein the tumor is a breast, colon, or stomach tumor.

31. The method of claim 26, wherein the antagonist of TYK2 is a small molecule antagonist.

32. The method of claim 26, wherein the antagonist of TYK2 is an antagonist antibody.

33. The method of claim 26, further comprising administering to the patient a cytotoxic agent, chemotherapeutic agent, or growth inhibitory agent.

34.-54. (canceled)


SEQUENCE LISTING (6 sequences)

SEQ ID NO: 1 - DNA, 4224 bp, Homo sapiens
gttcgagcttgtgttcccccggaagggtgagtctggacgcgggcgcggaaggagcgcggccggaggtcctcaggaagaagccgcggggactggctgcgcttgacaggctgcacttggatgggagcacctggtgcctcgggactgctccgatgcccgggtctgtgctgaatgtgtaatatgcggaactatattgaaacattacaaccatcttttgatggcaacaccctgaggacctcccttttccagatggggaaactgaggcccagaattgctaagtggcttgcttgagttgacacagggagctccaggactcaccctcagctgagccacctgccgggagcatgcctctgcgccactgggggatggccaggggcagtaagcccgttggggatggagcccagcccatggctgccatgggaggcctgaaggtgcttctgcactgggctggtccaggcggcggggagccctgggtcactttcagtgagtcatcgctgacagctgaggaagtctgcatccacattgcacataaagttggtatcactcctccttgcttcaatctctttgccctcttcgatgctcaggcccaagtctggttgcccccaaaccacatcctagagatccccagagatgcaagcctgatgctatatttccgcataaggttttatttccggaactggcatggcatgaatcctcgggaaccggctgtgtaccgttgtgggcccccaggaaccgaggcatcctcagatcagacagcacaggggatgcaactcctggacccagcctcatttgagtacctctttgagcagggcaagcatgagtttgtgaatgacgtggcatcactgtgggagctgtcgaccgaggaggagatccaccactttaagaatgagagcctgggcatggcctttctgcacctctgtcacctcgctctccgccatggcatccccctggaggaggtggccaagaagaccagcttcaaggactgcatcccgcgctccttccgccggcatatccggcagcacagcgccctgacccggctgcgccttcggaacgtcttccgcaggttcctgcgggacttccagccgggccgactctcccagcagatggtcatggtcaaatacctagccacactcgagcggctggcaccccgcttcggcacagagcgtgtgcccgtgtgccacctgaggctgctggcccaggccgagggggagccctgctacatccgggacagtggggtggcccctacagaccctggccctgagtctgctgctgggcccccaacccacgaggtgctggtgacaggcactggtggcatccagtggtggccagtagaggaggaggtgaacaaggaggagggttctagtggcagcagtggcaggaacccccaagccagcctgtttgggaagaaggccaaggctcacaaggcagtcggccagccggcagacaggccgcgggagccactgtgggcctacttctgtgacttccgggacatcacccacgtggtgctgaaagagcactgtgtcagcatccaccggcaggacaacaagtgcctggagctgagcttgccttcccgggctgcggcgctgtccttcgtgtcgctggtggacggctatttccgcctgacggccgactccagccactacctgtgccacgaggtggctcccccacggctggtgatgagcatccgggatgggatccacggacccctgctggagccatttgtgcaggccaagctgcggcccgaggacggcctgtacctcattcactggagcaccagccacccctaccgcctgatcctcacagtggcccagcgtagccaggcaccagacggcatgcagagcttgcggctccgaaagttccccattgagcagcaggacggggccttcgtgctggagggctggggccggtccttccccagcgttcgggaacttggggctgccttgcagggctgcttgctgagggccggggatgactgcttctctctgcgtcgctgttgcctgccccaaccaggagaaacctccaatctcatcatcatgcggggggctcgggccagccccaggacactcaacctcagccagctcagcttccaccgggttgaccagaaggagatcacccagctgtcccacttgggccagggcacaaggaccaacgtgtatgagggccgcctgcgagtggagggcagcggggaccctgaggagggcaagatggatgacgaggaccccctcgtgcctggcagggaccgtgggcaggagctacgagtggtgctcaaagtgctggaccctagtcaccatgacatcgccctggccttctacgagacagccagcctcatgagccaggtctcccacacgcacctggccttcgtgcatggcgtctgtgtgcgcggccctgaaaatatcatggtgacagagtacgtggagcacggacccctggatgtgtggctgcggagggagcggggccatgtgcccatggcttggaagatggtggtggcccagcagctggccagcgccctcagctacctggagaacaagaacctggttcatggtaatgtgtgtggccggaacatcctgctggcccggctggggttggcagagggcaccagccccttcatcaagctgagtgatcctggcgtgggcctgggcgccctctccagggaggagcgggtggagaggatcccctggctggcccccgaatgcctaccaggtggggccaacagcctaagcaccgccatggacaagtgggggtttggcgccaccctcctggagatctgctttgacggagaggcccctctgcagagccgcagtccctccgagaaggagcatttctaccagaggcagcaccggctgcccgagccctcctgcccacagctggccacactcaccagccagtgtctgacctatgagccaacccagaggccatcattccgcaccatcctgcgtgacctcacccggctgcagccccacaatcttgctgacgtcttgactgtgaacccggactcaccggcgtcggaccctacggttttccacaagcgctatttgaaaaagatccgagatctgggcgagggtcacttcggcaaggtcagcttgtactgctacgatccgaccaacgacggcactggcgagatggtggcggtgaaagccctcaaggcagactgcggcccccagcaccgctcgggctggaagcaggagattgacattctgcgcacgctctaccacgagcacatcatcaagtacaagggctgctgcgaggaccaaggcgagaagtcgctgcagctggtcatggagtacgtgcccctgggcagcctccgagactacctgccccggcacagcatcgggctggcccagctgctgctcttcgcccagcagatctgcgagggcatggcctatctgcactcgcagcactacatccaccgagacctagccgcgcgcaacgtgctgctggacaacgacaggctggtcaagatcggggactttggcctagccaaggccgtgcccgaaggccacgagtactaccgcgtgcgcgaggatggggacagccccgtgttctggtatgccccagagtgcctgaaggagtataagttctactatgcgtcagatgtctggtccttcggggtgaccctgtatgagctgctgacgcactgtgactccagccagagcccccccacgaaattccttgagctcataggcattgctcagggtcagatgacagttctgagactcactgagttgctggaacgaggggagaggctgccacggcccgacaaatgtccctgtgaggtctatcatctcatgaagaactgctgggagacagaggcgtcctttcgcccaaccttcgagaacctcatacccattctgaagacagtccatgagaagtaccaaggccaggccccttcagtgttcagcgtgtgctgaggcacaatggcagccctgcctgggaggactggaccaggcagtggctgcagagggagcctcctgctccctgctccaggatgaaaccaagagggggatgtcagcctcacccacaccgtgtgccttactcctgtctagagaccccacctctgtgaacttatttttctttcttggccgtgagcctaaccatgatcttgagggacccaacatttgtaggggcactaatccagcccttaaatcccccagcttccaaacttgaggcccaccatctccaccatctggtaataaactcatgttttctctgaaaaaaaaaaaaaaaaaa

SEQ ID NO: 2 - protein, 1187 aa, Homo sapiens
MPLRHWGMARGSKPVGDGAQPMAAMGGLKVLLHWAGPGGGEPWVTFSESSLTAEEVCIHIAHKVGITPPCFNLFALFDAQAQVWLPPNHILEIPRDASLMLYFRIRFYFRNWHGMNPREPAVYRCGPPGTEASSDQTAQGMQLLDPASFEYLFEQGKHEFVNDVASLWELSTEEEIHHFKNESLGMAFLHLCHLALRHGIPLEEVAKKTSFKDCIPRSFRRHIRQHSALTRLRLRNVFRRFLRDFQPGRLSQQMVMVKYLATLERLAPRFGTERVPVCHLRLLAQAEGEPCYIRDSGVAPTDPGPESAAGPPTHEVLVTGTGGIQWWPVEEEVNKEEGSSGSSGRNPQASLFGKKAKAHKAVGQPADRPREPLWAYFCDFRDITHVVLKEHCVSIHRQDNKCLELSLPSRAAALSFVSLVDGYFRLTADSSHYLCHEVAPPRLVMSIRDGIHGPLLEPFVQAKLRPEDGLYLIHWSTSHPYRLILTVAQRSQAPDGMQSLRLRKFPIEQQDGAFVLEGWGRSFPSVRELGAALQGCLLRAGDDCFSLRRCCLPQPGETSNLIIMRGARASPRTLNLSQLSFHRVDQKEITQLSHLGQGTRTNVYEGRLRVEGSGDPEEGKMDDEDPLVPGRDRGQELRVVLKVLDPSHHDIALAFYETASLMSQVSHTHLAFVHGVCVRGPENIMVTEYVEHGPLDVWLRRERGHVPMAWKMVVAQQLASALSYLENKNLVHGNVCGRNILLARLGLAEGTSPFIKLSDPGVGLGALSREERVERIPWLAPECLPGGANSLSTAMDKWGFGATLLEICFDGEAPLQSRSPSEKEHFYQRQHRLPEPSCPQLATLTSQCLTYEPTQRPSFRTILRDLTRLQPHNLADVLTVNPDSPASDPTVFHKRYLKKIRDLGEGHFGKVSLYCYDPTNDGTGEMVAVKALKADCGPQHRSGWKQEIDILRTLYHEHIIKYKGCCEDQGEKSLQLVMEYVPLGSLRDYLPRHSIGLAQLLLFAQQICEGMAYLHSQHYIHRDLAARNVLLDNDRLVKIGDFGLAKAVPEGHEYYRVREDGDSPVFWYAPECLKEYKFYYASDVWSFGVTLYELLTHCDSSQSPPTKFLELIGIAQGQMTVLRLTELLERGERLPRPDKCPCEVYHLMKNCWETEASFRPTFENLIPILKTVHEKYQGQAPSVFSVC

SEQ ID NO: 3 - DNA, 5745 bp, Homo sapiens
gtggcctgcccaacgtgtgctgggtgggagaaggcgaggcggcagcgatgctgtctcttccgtgaggagcgcagaggaggtcgcggcgccggaggccccagaaggctcgaaggcgccgcgggctggggtcggtggcttagggagcccgtccggccatggtggccgcgggtggtggttggcgcggctgcgctgcggcccggggcagtgcggagccgggacagtcgcggcgctgacgcccgcgggccccagctgcagatatgaagcggagccgctgccgcgaccgaccgcagccgccgccgcccgaccgccgggaggatggagttcagcgggcagcggagctgtctcagtctttgccgccgcgccggcgagcgccgcccgggaggcagcggctggaggagcggacgggccccgcggggcccgagggcaaggagcaggatgtagtaactggagttagtcccctgctcttcaggaaactcagtaatcctgacatattttcatccactggaaaagttaaacttcagcgacaactgagtcaggatgattgtaagttatggagaggaaacctggccagctctctatcgggtaagcagctgctccctttgtccagcagtgtacatagcagtgtgggacaggtgacttggcagtcgtcaggagaagcatcaaacctggttcgaatgagaaaccagtcccttggacagtctgcaccttctcttactgctggcctgaaggagttgagccttccaagaagaggcagcttttgtcggacaagtaaccgcaagagcttgattgtgacctctagcacatcacctacactaccacggccacactcaccactccatggccacacaggtaacagtcctttggacagcccccggaatttctctccaaatgcacctgctcacttttcttttgttcctgcccgtaggactgatgggcggcgctggtctttggcctctttgccctcttcaggatatggaactaacactcctagctccactgtctcatcatcatgctcctcacaggaaaagctgcatcagttgcctttccagcctacagctgatgagctgcactttttgacgaagcatttcagcacagagagcgtaccagatgaggaaggacggcagtccccagccatgcggcctcgctcccggagcctcagtcccggacgatccccagtatcctttgacagtgaaataataatgatgaatcatgtttacaaagaaagattcccaaaggccaccgcacaaatggaagagcgactagcagagtttatttcctccaacactccagacagcgtgctgcccttggcagatggagccctgagctttattcatcatcaggtgattgagatggcccgagactgcctggataaatctcggagtggcctcattacatcacaatacttctacgaacttcaagataatttggagaaacttttacaagatgctcatgagcgctcagagagctcagaagtggcttttgtgatgcagctggtgaaaaagctgatgattatcattgcccgcccagcacgtctcctggaatgcctggagtttgaccctgaagagttctaccaccttttagaagcagctgagggccacgccaaagagggacaagggattaaatgtgacattccccgctacatcgttagccagctgggcctcacccgggatcccctagaagaaatggcccagttgagcagctgtgacagtcctgacactccagagacagatgattctattgagggccatggggcatctctgccatctaaaaagacaccctctgaagaggacttcgagaccattaagctcatcagcaatggcgcctatggggctgtatttctggtgcggcacaagtccacccggcagcgctttgccatgaagaagatcaacaagcagaacctgatcctacggaaccagatccagcaggccttcgtggagcgtgacatactgactttcgctgagaacccctttgtggtcagcatgttctgctcctttgataccaagcgccacttgtgcatggtgatggagtacgttgaagggggagactgtgccactctgctgaagaatattggggccctgcctgtggacatggtgcgtctatactttgcggaaactgtgctggccctggagtacttacacaactatggcatcgtgcaccgtgacctcaagcctgacaacctcctaattacatccatggggcacatcaagctcacggactttggactgtccaaaattggcctcatgagtctgacaacgaacttgtatgagggtcatattgaaaaggatgcccgggaattcctggacaagcaggtatgcgggaccccagaatacattgcgcctgaggtgatcctgcgccagggctatgggaagccagtggactggtgggccatgggcattatcctgtatgagttcctggtgggctgcgtccctttttttggagatactccggaggagctctttgggcaggtgatcagtgatgagattgtgtggcctgagggtgatgaggcactgcccccagacgcccaggacctcacctccaaactgctccaccagaaccctctggagagacttggcacaggcagtgcctatgaggtgaagcagcacccattctttactggtctggactggacaggacttctccgccagaaggctgaatttattcctcagttggagtcagaggatgatactagctattttgacacccgctcagagcgataccaccacatggactcggaggatgaggaagaagtgagtgaggatggctgccttgagatccgccagttctcttcctgctctccaaggttcaacaaggtgtacagcagcatggagcggctctcactgctcgaggagcgccggacaccacccccgaccaagcgcagcctgagtgaggagaaggaggaccattcagatggcctggcagggctcaaaggccgagaccggagctgggtgattggctcccctgagatattacggaagcggctgtcggtgtctgagtcgtcccacacagagagtgactcaagccctccaatgacagtgcgacgccgctgctcaggcctcctggatgcgcctcggttcccggagggccctgaggaggccagcagcaccctcaggaggcaaccacaggagggtatatgggtcctgacacccccatctggagagggggtatctgggcctgtcactgaacactcaggggagcagcggccaaagctggatgaggaagctgttggccggagcagtggttccagtccagctatggagacccgaggccgtgggacctcacagctggctgagggagccacagccaaggccatcagtgacctggctgtgcgtagggcccgccaccggctgctctctggggactcaacagagaagcgcactgctcgccctgtcaacaaagtgatcaagtccgcctcagccacagccctctcactcctcattccttcggaacaccacacctgctccccgttggccagccccatgtccccacattctcagtcgtccaacccatcatcccgggactcttctccaagcagggacttcttgccagcccttggcagcatgaggcctcccatcatcatccaccgagctggcaagaagtatggcttcaccctgcgggccattcgcgtctacatgggtgactccgatgtctacaccgtgcaccatatggtgtggcacgtggaggatggaggtccggccagtgaggcagggcttcgtcaaggtgacctcatcacccatgtcaatggggaacctgtgcatggcctggtgcacacggaggtggtggagctgatcctgaagagtggaaacaaggtggccatttcaacaactcccctggagaacacatccattaaagtggggccagctcggaagggcagctacaaggccaagatggcccgaaggagcaagaggagccgcggcaaggatgggcaagaaagaaaaaggagctccctgttccgcaagatcaccaagcaagcatccctgctccacaccagccgcagcctttcttcccttaaccgctccttgtcatcaggggagagtgggccaggctctcccacacacagccacagcctttccccccgatctcccactcaaggctaccgggtgacccccgatgctgtgcattcagtgggagggaattcatcacagagcagctcccccagctccagcgtgcccagttccccagccggctctgggcacacacggcccagctccctccacggtctggcacccaagctccaacgccagtaccgctctccacggcgcaagtcagcaggcagcatcccactgtcaccactggcccacaccccttctcccccacccccaacagcttcacctcagcggtccccatcgcccctgtctggccatgtagcccaggcctttcccacaaagcttcacttgtcacctcccctgggcaggcaactctcacggcccaagagtgcggagccaccccgttcaccactactcaagagggtgcagtcggctgagaaactggcagcagcacttgccgcctctgagaagaagctagccacctctcgcaagcacagccttgacctgccccactctgaactaaagaaggaactgccgcccagggaagtgagccctctggaggtagttggagccaggagtgtgctgtctggcaagggggccctgccagggaagggggtgctgcagcctgctccctcacgggccctaggcaccctccggcaggaccgagccgaacgacgggagtcgctgcagaagcaagaagccattcgtgaggtggactcctcagaggacgacaccgaggaagggcctgagaacagccagggtgcacaggagctgagcttggcacctcacccagaagtgagccagagtgtggcccctaaaggagcaggagagagtggggaagaggatcctttcccgtccagagaccctaggagcctgggcccaatggtcccaagcctattgacagggatcacactggggcctcccagaatggaaagtcccagtggtccccacaggaggctcgggagcccacaagccattgaggaggctgccagctcctcctcagcaggccccaacctaggtcagtctggagccacagaccccatccctcctgaaggttgctggaaggcccagcacctccacacccaggcactaacagcactttctcccagcacttcgggactcacccccaccagcagttgctctcctcccagctccacctctgggaagctgagcatgtggtcctggaaatcccttattgagggcccagacagggcatccccaagcagaaaggcaaccatggcaggtgggctagccaacctccaggatttggaaaacacaactccagcccagcctaagaacctgtctcccagggagcaggggaagacacagccacctagtgcccccagactggcccatccatcttatgaggatcccagccagggctggctatgggagtctgagtgtgcacaagcagtgaaagaggatccagccctgagcatcacccaagtgcctgatgcctcaggtgacagaaggcaggacgttccatgccgaggctgccccctcacccagaagtctgagcccagcctcaggaggggccaagaaccagggggccatcaaaagcatcgggatttggcattggttccagatgagcttttaaagcaaacatagcagttgtttgccatttcttgcactcagacctgtgtaatatatgctcctggaaaccaaaaaaaaaaaaaaaaaaaaaaaaaaaaaaaaaaaaaaa

SEQ ID NO: 4 - protein, 1798 aa, Homo sapiens
MKRSRCRDRPQPPPPDRREDGVQRAAELSQSLPPRRRAPPGRQRLEERTGPAGPEGKEQDVVTGVSPLLFRKLSNPDIFSSTGKVKLQRQLSQDDCKLWRGNLASSLSGKQLLPLSSSVHSSVGQVTWQSSGEASNLVRMRNQSLGQSAPSLTAGLKELSLPRRGSFCRTSNRKSLIVTSSTSPTLPRPHSPLHGHTGNSPLDSPRNFSPNAPAHFSFVPARRTDGRRWSLASLPSSGYGTNTPSSTVSSSCSSQEKLHQLPFQPTADELHFLTKHFSTESVPDEEGRQSPAMRPRSRSLSPGRSPVSFDSEIIMMNHVYKERFPKATAQMEERLAEFISSNTPDSVLPLADGALSFIHHQVIEMARDCLDKSRSGLITSQYFYELQDNLEKLLQDAHERSESSEVAFVMQLVKKLMIIIARPARLLECLEFDPEEFYHLLEAAEGHAKEGQGIKCDIPRYIVSQLGLTRDPLEEMAQLSSCDSPDTPETDDSIEGHGASLPSKKTPSEEDFETIKLISNGAYGAVFLVRHKSTRQRFAMKKINKQNLILRNQIQQAFVERDILTFAENPFVVSMFCSFDTKRHLCMVMEYVEGGDCATLLKNIGALPVDMVRLYFAETVLALEYLHNYGIVHRDLKPDNLLITSMGHIKLTDFGLSKIGLMSLTTNLYEGHIEKDAREFLDKQVCGTPEYIAPEVILRQGYGKPVDWWAMGIILYEFLVGCVPFFGDTPEELFGQVISDEIVWPEGDEALPPDAQDLTSKLLHQNPLERLGTGSAYEVKQHPFFTGLDWTGLLRQKAEFIPQLESEDDTSYFDTRSERYHHMDSEDEEEVSEDGCLEIRQFSSCSPRFNKVYSSMERLSLLEERRTPPPTKRSLSEEKEDHSDGLAGLKGRDRSWVIGSPEILRKRLSVSESSHTESDSSPPMTVRRRCSGLLDAPRFPEGPEEASSTLRRQPQEGIWVLTPPSGEGVSGPVTEHSGEQRPKLDEEAVGRSSGSSPAMETRGRGTSQLAEGATAKAISDLAVRRARHRLLSGDSTEKRTARPVNKVIKSASATALSLLIPSEHHTCSPLASPMSPHSQSSNPSSRDSSPSRDFLPALGSMRPPIIIHRAGKKYGFTLRAIRVYMGDSDVYTVHHMVWHVEDGGPASEAGLRQGDLITHVNGEPVHGLVHTEVVELILKSGNKVAISTTPLENTSIKVGPARKGSYKAKMARRSKRSRGKDGQESRKRSSLFRKITKQASLLHTSRSLSSLNRSLSSGESGPGSPTHSHSLSPRSPTQGYRVTPDAVHSVGGNSSQSSSPSSSVPSSPAGSGHTRPSSLHGLAPKLQRQYRSPRRKSAGSIPLSPLAHTPSPPPPTASPQRSPSPLSGHVAQAFPTKLHLSPPLGRQLSRPKSAEPPRSPLLKRVQSAEKLAAALAASEKKLATSRKHSLDLPHSELKKELPPREVSPLEVVGARSVLSGKGALPGKGVLQPAPSRALGTLRQDRAERRESLQKQEAIREVDSSEDDTEEGPENSQGAQELSLAPHPEVSQSVAPKGAGESGEEDPFPSRDPRSLGPMVPSLLTGITLGPPRMESPSGPHRRLGSPQAIEEAASSSSAGPNLGQSGATDPIPPEGCWKAQHLHTQALTALSPSTSGLTPTSSCSPPSSTSGKLSMWSWKSLIEGPDRASPSRKATMAGGLANLQDLENTTPAQPKNLSPREQGKTQPPSAPRLAHPSYEDPSQGWLWESECAQAVKEDPALSITQVPDASGDRRQDVPCRGCPLTQKSEPSLRRGQEPGGHQKHRDLALVPDELLKQT

SEQ ID NO: 5 - DNA, 1832 bp, Homo sapiens
gctttgcggctgtcgtcggagaggcatctgggttcggactggggccgccatggggaaagtgaatgtggccaagttgcgttacatgagccgagatgacttcagggtcttgaccgcggttgaaatgggcatgaagaaccatgaaattgttcccggcagtttgattgcttctatagccagccttaaacatggtggctgtaataaagttttaagagaattagtgaaacataaactcatagcttgggagcgtaccaaaactgtccagggctatcggttgacaaatgcaggatatgattacctagctttgaaaacactttcttctaggcaagtagttgagtctgttggaaaccagatgggtgttggcaaagaatcagatatttacattgttgcaaatgaagaaggacaacaatttgcattaaagcttcacagactaggaagaacctcgtttcgaaatttgaaaaacaaacgcgattatcataaacataggcacaatgtgtcttggctttatttatctcgtctctctgccatgaaggaatttgcctatatgaaggcattgtatgagaggaaatttccagttccaaagccaattgattacaatcgtcatgcagtggtcatggaactcataaatggttatccactatgtcagatacaccatgttgaagatcctgcatcagtatatgatgaagctatggaactaattgtcaaacttgcaaatcatgggctgattcatggagattttaatgaatttaatctcattttggatgaaagtgaccatatcaccatgattgattttccacagatggtttcaacttctcatcccaatgctgagtggtattttgacagagatgttaaatgcattaaagatttctttatgaaacgtttcagctacgaaagtgagctttttccaacttttaaggatatcaggagagaagacactcttgatgtggaggtttctgccagtggctacacaaaggaaatgcaggcagatgatgaactgcttcatccattaggtccagatgataaaaatattgaaacaaaagagggatctgaattctcattttcagatggagaagtggcagaaaaagcagaggtttacaggtcagaaaatgaaagtgaacggaactgtctagaagaatcagagggctgctattgcagatcatctggagaccctgaacaaataaaggaagacagtttatcagaagagagtgctgatgcacggagttttgaaatgactgaattcaatcaagctttagaagaaataaaagggcaggttgttgaaaacaactctgtaactgaattttctgaggagaaaaacagaactgaaaattacaacaggcaagatggtcagagagttcaaggaggagtccctgctggctctgacgagtatgaagatgaatgccctcatctaattgccttgtcgtcattaaatagagaattcaggcctttcagagatgaagaaaatgtgggagctatgaatcagtatagaacaagaactctgagtatcacttcttcaggcagtgctgtaagctgttcaacaattcctccagaactggtgaaacagaaggtgaaacgtcagttgacaaaacagcaaaaatcagctgtcagacgtcgattgcagaaaggagaagcaaatatatttaccaagcaacgtagggaaaacatgcaaaatatcaaatcaagtttggaagcagctagcttttggggagaataatatatttaggatcttggatatgtttaatatattttttaaagttactgtaattcctttttgagccctcatttgtcttttttgagccaaggctatcatatattaataaataaaccctctttcatct

SEQ ID NO: 6 - protein, 552 aa, Homo sapiens [sequence omitted]